(19) United States
(12) Patent Application Publication
(10) Pub. No.: US 2023/0364134 A1
(43) Pub. Date: Nov. 16, 2023

(54) METHOD FOR IMPROVING HEALTH CONDITIONS

(71) Applicant: NISSIN FOODS HOLDINGS CO., LTD., Osaka-shi, Osaka (JP)

(72) Inventors: Noritaka ANDO, Osaka (JP); Takuo NAKAZEKO, Osaka (JP); Yukio HIRANO, Osaka (JP); Futoshi NAKAMURA, Osaka (JP)

(21) Appl. No.: 18/246,800

(22) PCT Filed: Sep. 30, 2021
(86) PCT No.: PCT/JP2021/036072

§ 371 Date: Mar. 27, 2023

(30) Foreign Application Priority Data
Oct. 1, 2020 (JP) 2020-166818

(51) Int. Cl. A61K 33/42; A61K 31/07; A61K 31/51; A61K 31/375; A61K 31/525; A61K 31/4415; A61K 31/519; A61K 33/00; A61K 33/06; A61K 33/26; A61P 3/02; A23L 33/17; A23L 33/12; A23L 33/21; A23L 33/15; A23L 33/16; A23L 33/155; A23L 33/00

(52) U.S. Cl. CPC A61K 33/42; A61K 31/07; A61K 31/51; A61K 31/375; A61K 31/525; A61K 31/4415; A61K 31/519; A61K 33/00; A61K 33/06; A61K 33/26; A61P 3/02; A23L 33/17; A23L 33/12; A23L 33/21; A23L 33/15; A23L 33/16; A23L 33/155; A23L 33/40

(57) ABSTRACT

A method for improving health conditions is developed by studying the way meals containing adjusted nutritional components in terms of not only the content of the meal, but also time, period, and the like when the meals are taken. The present invention improves the health conditions by continually taking at least one meal selected from daily three meals, i.e., breakfast, lunch, and evening meal, which contains 13% to 20% of proteins, 20% to 30% of fats, and 50% to 65% of carbohydrates in terms of energy percentage and in which proteins, fats, carbohydrates, dietary fiber, vitamins, and minerals are adjusted to predetermined amounts.

### TECHNICAL FIELD

The present invention relates to a method for improving health conditions by taking an adjusted meal.

### BACKGROUND ART

In recent years, increases in medical and care costs have become problematic with the aging of the population. In ensuring less medical spending and comfortable retirement, it is an important challenge to extend a “health span,” which is the length of time when one can live without daily life restrictions due to health issues. For example, the Ministry of Health, Labour and Welfare of Japan revises the standards of recommended energy and nutrient intakes to maintain and promote the public health every five years, and published a report from the committee for developing and reviewing the “2020 Dietary Reference Intakes for Japanese” (hereinafter, the report is referred to as “Dietary Reference Intakes for Japanese”) on Dec. 24, 2019.

In the “Dietary Reference Intakes for Japanese,” the tentative dietary goals for preventing life-style related diseases for the three major nutrients, proteins, fats, and carbohydrates (proportion of each nutrient to total energy intake) are established depending on the estimated energy requirement estimated energy requirement (kcal/day) calculated for each category of age, sex, and physical activity level (low [I], moderate [II], or high [III]). The recommended dietary allowance, adequate intake, tolerable upper intake level, or tentative dietary goal for preventing life-style related diseases or a combination thereof is established for lipophilic vitamins such as vitamin A and vitamin D, water-soluble vitamins such as vitamin B1, vitamin B2, and niacin, and minerals such as sodium, potassium, calcium, magnesium, phosphorus, iron, zinc, and manganese.

The reference values such as the recommended dietary allowance, adequate intake, tolerable upper intake level, and tentative dietary goal for preventing life-style related diseases for each nutrient in the “Dietary Reference Intakes for Japanese” are established for each category of sex, age, and physical activity level. Trial and error are required to calculate nutrition so that all nutrients should meet the reference values, and nutrition is often calculated so that the three major nutrients and the salt equivalent alone or main vitamins and main minerals in addition thereto should meet the reference values. Further, nutrition is not calculated so that nutrients in each meal should meet the reference values, but nutrition is often calculated so that average nutritional intakes in an individual should meet the reference values in a specific period, such as one week, for example.

Patent Literature 1 describes an invention relating to a nutrition-adjusted food product for oral ingestion which comprises at least 2% to 75% of carbohydrates, 10% or higher of proteins, and 15% to 70% of fats in terms of energy percentage, and is designed so that the vitamin and mineral intakes established in the “Dietary Reference Intakes for Japanese” issued by the Ministry of Health, Labour and Welfare of Japan should be not less than the requirements and not more than the upper-limit amounts when the estimated energy requirement established in the Dietary Reference Intakes for Japanese issued by the Ministry of Health, Labour and Welfare of Japan is ingested.

### CITATION LIST

### Patent Literature

### Patent Literature 1

Japanese Patent Laid-open No. 2019-140952

Patent Literature 1 discloses an invention relating to a method for taking a meal with adjusted nutritional components, with an object of improvement of health conditions described above. Meanwhile, it is assumed that there are various methods other than that disclosed in Patent Literature 1.

When a meal with adjusted nutritional components is taken, it is considered that the outcome is often dependent on various factors such as the content of the meal as well as the timing of taking the meal (i.e., breakfast, lunch, or evening meal) and the duration of the period when the meal is continually taken. However, not much specific data showing the effect of continually taking such an adjusted meal are available at this point.

### SUMMARY OF INVENTION

### Technical Problem

Accordingly, the present inventors have studied a new method for taking a meal with adjusted nutritional components, in terms of not only the content of the meal to be taken, but also the time, timing, and others when the meal is taken. The present inventors have further studied the effect of actually taking the adjusted meal for the purpose of improving health conditions.

### Solution to Problem

As a result of various studies, the present inventors have found that health conditions are improved by continually taking at least two meals selected from daily three meals, i.e., breakfast, lunch, and evening meal, which contain 13% to 20% of proteins, 20% to 30% of fats, and 50% to 65% of carbohydrates in terms of energy percentage and in which proteins, fats, carbohydrates, dietary fiber, vitamins, and minerals are adjusted to predetermined amounts, and thus the present invention was accomplished.

Specifically, the first invention of the present application is

“a method for improving health conditions by continually taking at least one meal selected from daily three meals, i.e., breakfast, lunch, and evening meal, which contains 13% to 20% of proteins, 20% to 30% of fats, and 50% to 65% of carbohydrates in terms of energy percentage and in which proteins, fats, carbohydrates, dietary fiber, vitamins, and minerals are adjusted to predetermined amounts.”

Further, the health conditions are preferably assessed by any one or more health indicators selected from body weight, BMI, body fat percentage, blood pressure, blood triglyceride, bone density, presenteeism, oxidative stress marker, stool frequency, and intestinal flora diversity.

That is, the second invention of the present application is

“the method for improving health conditions according to claim 1, wherein the health conditions are assessed by any one or more health indicators selected from body weight, BMI, body fat percentage, blood pressure, blood triglyceride, bone density, presenteeism, oxidative stress marker, stool frequency, and intestinal flora diversity.”

Further, it is preferable that the vitamins include at least vitamin B1 and vitamin C, and the minerals include at least Ca, Mg, and Fe.

That is, the third invention of the present application is

“the method for improving health conditions according to claim 1 or 2, wherein the vitamins include at least vitamin B1 and vitamin C, and the minerals include at least Ca, Mg, and Fe.”

Further, it is preferable that the vitamins include at least vitamin A, vitamin B1, vitamin B2, vitamin B6, folic acid, and vitamin C, and the minerals include at least K, Ca, Mg, P, and Fe.

That is, the fourth invention of the present application is

“the method for improving health conditions according to claim 1 or 2, wherein the vitamins include at least vitamin A, vitamin B1, vitamin B2, vitamin B6, folic acid, and vitamin C, and the minerals include at least K, Ca, Mg, P, and Fe.”

Further, it is preferable that the fats include saturated fatty acids not more than a predetermined amount and n-3 fatty acids and n-6 fatty acids not less than predetermined amounts.

That is, the fifth invention of the present application is

“the method for improving health conditions according to any of claims 1 to 4, wherein the fats include saturated fatty acids not more than a predetermined amount and n-3 fatty acids and n-6 fatty acids not less than predetermined amounts.”

Further, the meal may be taken continually only on weekdays.

That is, the sixth invention of the present application is

“the method for improving health conditions according to any of claims 1 to 5, wherein the meal is taken continually only on weekdays.”

Further, the meal is preferably taken continually for at least 3 weeks. That is, the seventh invention of the present application is

“the method for improving health conditions according to any of claims 1 to 6, wherein the meal is taken continually for at least 3 weeks.”

Further, the present applicants intend that the present invention also encompasses a meal with adjusted nutritional components to be taken by the method for improving health conditions according to any of the first to seventh inventions.

That is, the eighth invention of the present application is

“a meal with adjusted nutritional components to be taken in the method for improving health conditions according to any of claims 1 to 7.”

Further, the meal is at least one meal selected from the group consisting of breakfast, lunch, and evening meal.

That is, the ninth invention of the present application is

“the meal according to claim 8, wherein the meal is at least one meal selected from the group consisting of breakfast, lunch, and evening meal.”

### Advantageous Effects of Invention

When the method of the present invention is used, the health conditions can be improved by continually taking a meal with adjusted nutritional components. In particular, the health conditions assessed by any one or more health indicators selected from blood triglyceride, bone density, presenteeism, oxidative stress marker, and intestinal flora diversity can be improved.

### BRIEF DESCRIPTION OF DRAWINGS

FIG. 1A and FIG. 1B are tables showing the estimated energy requirement and the tentative dietary goals for preventing life-style related diseases, etc. for proteins, fats, saturated fatty acids, n-3 fatty acids, n-6 fatty acids, and carbohydrates at each physical activity level for 18 to 64 years of age in the “Dietary Reference Intakes for Japanese” relating to the present invention.

FIG. 2 is a table showing the results of physical measurements before and after continually taking the adjusted meal in the example of the present invention for a predetermined period.

FIG. 3 is a table showing the results of analyzing levels of triglyceride among blood lipids before and after continually taking the adjusted meal in the example of the present invention for a predetermined period.

FIG. 4 is a table showing the results of analyzing levels of 8-hydroxy-2′-deoxyguanosine (8-OHdG) among metabolites in blood (plasma) of subjects by capillary-electrophoresis-time-of-flight mass spectrometry (CE-TOFMS) for the analysis of blood metabolome before and after continually taking the adjusted meal in the example of the present invention for a predetermined period.

FIG. 5 is a table showing the results of measuring blood pressure before and after continually taking the adjusted meal in the example of the present invention for a predetermined period.

FIG. 6 is a table showing the results of measuring bone density before and after continually taking the adjusted meal in the example of the present invention for a predetermined period.

FIG. 7 is a table showing the results of analyzing the intestinal flora before and after continually taking the adjusted meal in the example of the present invention for a predetermined period.

### DESCRIPTION OF EMBODIMENTS

The present invention relates to the following. The present invention will be described in detail below.

The present invention relates to “a method for improving health conditions by continually taking at least one meal selected from daily three meals, i.e., breakfast, lunch, and evening meal, which contains 13% to 20% of proteins, 20% to 30% of fats, and 50% to 65% of carbohydrates in terms of energy percentage and in which proteins, fats, carbohydrates, dietary fiber, vitamins, and minerals are adjusted to predetermined amounts.”

The present invention will be described in detail below.

—Time of Taking a Meal with Adjusted Nutritional Components in Terms of Daily Three Meals—

In the present invention, a meal with adjusted nutritional components described later is taken for at least one meal from daily breakfast, lunch, and evening meal. Further, the meal is preferably taken for at least two meals from daily breakfast, lunch, and evening meal. Specifically, a combination of breakfast and lunch, breakfast and evening meal, or lunch and evening meal can be selected in this case. Further, it is more preferable that the adjusted meal is taken for all breakfast, lunch, and evening meal.

Further, the meal with adjusted nutritional components according to the present invention is preferably taken particularly for a meal with high energy intake from breakfast, lunch, and evening meal.

### —Amount of Energy—

During the period when the adjusted meal of the present invention is taken, the total amount of energy intake in daily breakfast, lunch, and evening meal is not particularly limited and preferably meets the Dietary Reference Intakes for Japanese (Dec. 24, 2019).

In other words, the Ministry of Health, Labour and Welfare of Japan revises the standards for recommended energy and nutrient intakes to maintain and promote the public health every five years, and published a report from the committee for developing and reviewing the “2020 Dietary Reference Intakes for Japanese” (hereinafter, the report is referred to as “Dietary Reference Intakes for Japanese”) on Dec. 24, 2019.

The “Dietary Reference Intakes for Japanese” discloses the estimated energy requirement (kcal/day) for each category of age, sex, and physical activity level (low [I], moderate [II], high [III]), and it is preferable to meet the estimated energy requirements.

The amount of energy can be set variously depending on the category such as the subject's sex or age, and it can be 1700 kcal or 2000 kcal depending on the subject or age.

Further, examples of the energy intake ratio for breakfast, lunch, and evening meal (including between-meal snacks) in the present invention include approximately 20:30:50 for breakfast:lunch:evening meal (including between-meal snacks).

However, the energy intake ratio is not limited to this example and is preferably roughly within a range of 10% to 30%:20% to 40%:40% to 60% for breakfast:lunch:evening meal.

Here, when the meal with adjusted nutritional components is taken for one meal from daily breakfast, lunch, and evening meal, any of breakfast, lunch, and evening meal can be selected, but the meal with adjusted nutritional components is preferably a meal with high calorie intake (lunch or evening meal).

Further, when the meal with adjusted nutritional components is taken for two meals in a day, the total calorie intake in these meals with adjusted nutritional components is preferably 35% or more of the total daily calorie intake, more preferably 45% or more, yet more preferably 50% or more.

In other words, the proportion of the number of calories taken in the meals with the adjusted meal (two meals selected from breakfast, lunch, and evening meal) to the total number of calories taken in a day is preferably high. Of note, the total number of calories taken in a day is preferably designed beforehand.

Further, when the adjusted meal described later is eaten for one meal in a day, the meal content for the remaining two meals is not particularly limited.

Further, when the adjusted meal described later is eaten for two meals in a day, the meal content for the remaining one meal is not particularly limited as long as the adjusted meal described later is eaten for two meals in a day.

However, the meal content for meals other than the adjusted meal is preferably nutritionally balanced as in the adjusted meal.

### —Energy Percentage—

The meal with adjusted nutritional components of the present invention is a meal with adjusted nutritional components containing 13% to 20% proteins, 20% to 30% fats, and 50% to 65% carbohydrates in terms of energy percentage, that is, an adjusted meal with PFC balance.

This balance of intakes of proteins, fats, and carbohydrates is described as energy-yielding nutrient balance in the 2020 Dietary Reference Intakes for Japanese.

### —Proteins—

Proteins are taken in consideration of the above-described PFC balance, and protein intake is preferably determined within a range between the values obtained by preferably setting the upper limit and the lower limit of total daily protein intake as described below and multiplying these upper limit and lower limit by the value obtained by dividing the number of calories taken in a meal to be eaten (breakfast, lunch, or evening meal) by the total number of calories designed to be taken in a day (i.e., the percentage of calorie intake of each meal).

Here, the upper limit and lower limit of total daily protein intake are preferably calculated as described below.

In the “Dietary Reference Intakes for Japanese,” 13% to 20% or 14% to 20% of energy per day is established as the Dietary Reference Intakes (a tentative dietary goal for preventing life-style related diseases) of proteins for men and women aged 18 to 64 years. Additionally, 65 g/day and 50 g/day are established as the recommended dietary allowance for men and women, respectively. Although the lower limit can be lower than the recommended dietary allowance for a person who requires low energy intake, the “Dietary Reference Intakes for Japanese” recommends setting the lower limit not lower than the recommended dietary allowance even in such a case.

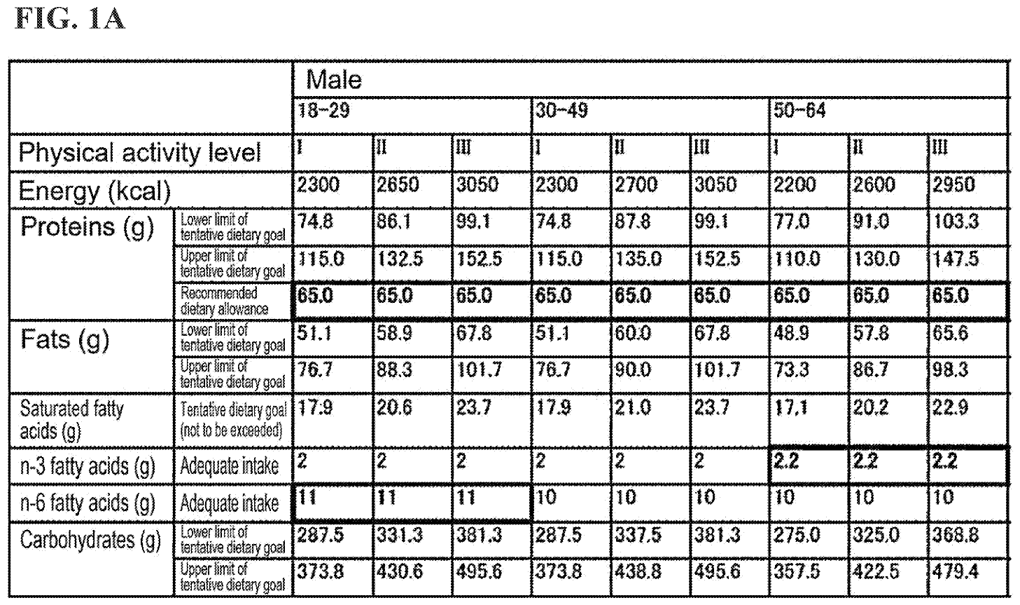

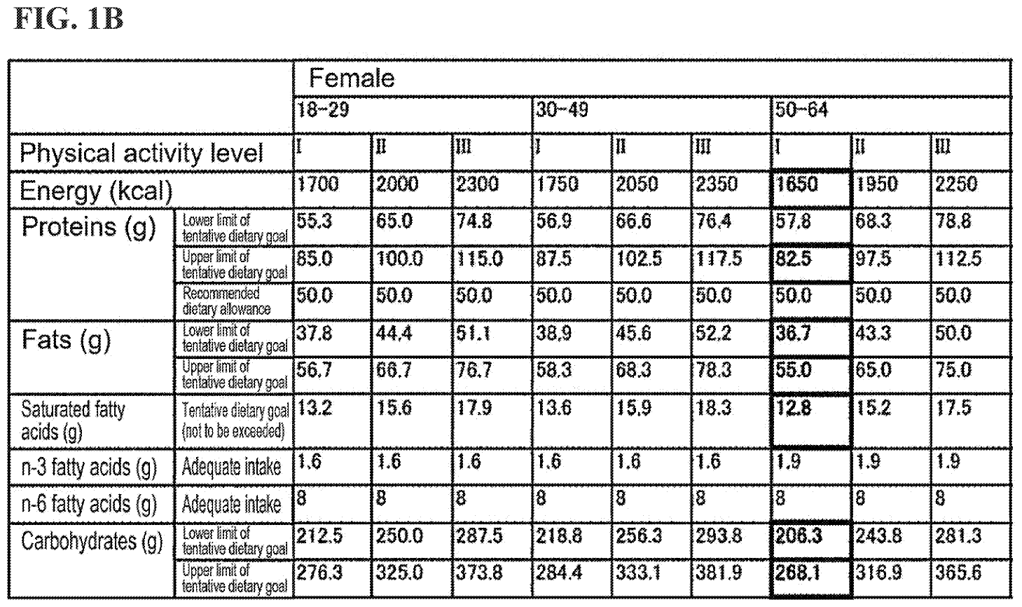

Since the estimated energy requirement varies for each category of sex, age, and physical activity level, the upper limit and the lower limit of the tentative dietary goal for preventing life-style related diseases for proteins vary depending on the category of the Dietary Reference Intakes when the upper limit and the lower limit are calculated in g/day as shown in FIGS. 1A and 1B.

Accordingly, the upper limit of protein intake in the adjusted meal is defined as the upper limit of the tentative dietary goal for preventing life-style related diseases for proteins in the “reference amount of energy” category with the lowest estimated energy requirement, so that users with different sex, age, and physical activity level can achieve the Dietary Reference Intakes for Japanese. Further, the lower limit of protein intake in the adjusted meal is defined as the highest recommended dietary allowance in the category to which each user belongs.

In the example shown in FIGS. 1A and 1B, the upper limit of protein intake in the adjusted meal is the upper limit of the tentative dietary goal for preventing life-style related diseases for protein in the category with the lowest estimated energy requirement, i.e., the category of female, 50 to 64 years of age, and physical activity level I (low). In this case, the upper limit of protein intake in the adjusted meal is 82.5 g/day (=1650 kcal/4 kcal×20%) since 1 g of protein provides 4 kcal of energy in the body. Meanwhile, the lower limit of protein intake is 60 g, the highest recommended dietary allowance among all categories.

Further, as another adjusting method, it is also preferable that protein intake is determined within a range between the values obtained by defining the above-mentioned number of calories taken in a day as 1650 kcal in the category with the lowest estimated energy requirement, i.e., the category of female, 50 to 64 years of age, and physical activity level I (low), in the Dietary Reference Intakes for Japanese for 18 to 64 years of age shown in FIGS. 1A and 1B and multiplying the above-mentioned upper limit and lower limit of protein intake (82.5 g/day and 60 g/day) by the value obtained by dividing the number of calories taken in a meal with adjusted nutritional components to be eaten (breakfast, lunch, or evening meal) by the above-mentioned 1650 kcal.

### —Fats—

Fats are taken in consideration of the PFC balance, and fat intake is preferably determined within a range between the values obtained by preferably setting the upper limit and the lower limit of total daily fat intake as described below and multiplying these upper limit and lower limit by the value obtained by dividing the number of calories taken in a meal to be eaten (breakfast, lunch, or evening meal) by the total number of calories designed to be taken in a day (i.e., percentage of calorie intake of each meal).

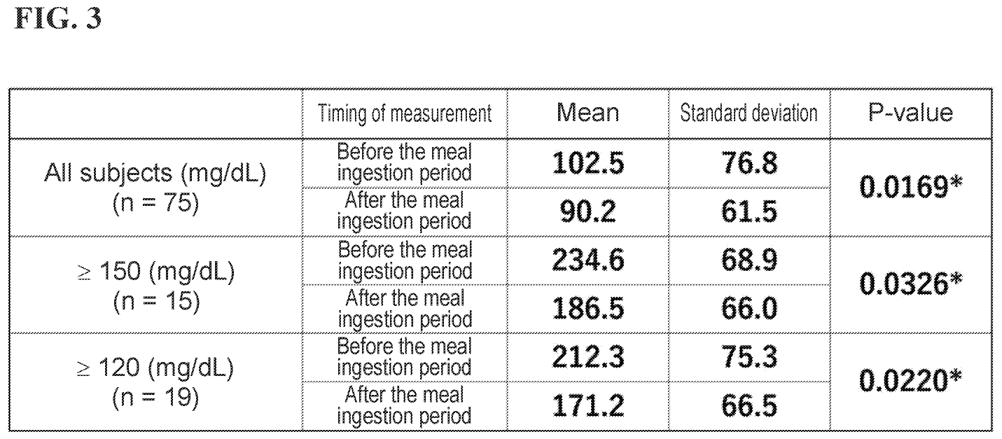

Here, the upper limit and lower limit of total daily fat intake can be calculated as described below. In the “Dietary Reference Intakes for Japanese,” 20% to 30% of energy per day is established as the Dietary Reference Intakes (a tentative dietary goal for preventing life-style related diseases) of fats for men and women aged 18 to 64 years. Since the estimated energy requirement varies for each category of sex, age, and physical activity level, the upper limit and the lower limit of the tentative dietary goal for preventing life-style related diseases for fats vary depending on the category as shown in FIG. 3.

Accordingly, the upper limit of fat intake in the adjusted meal is defined as the upper limit of the tentative dietary goal for preventing life-style related diseases for fats in the “reference amount of energy” category with the lowest estimated energy requirement, so that users with different sex, age, and physical activity level can eat a nutritionally complete diet that meets the Dietary Reference Intakes for Japanese.

Meanwhile, since the recommended dietary allowance of fats is not established in the “Dietary Reference Intakes for Japanese,” the lower limit of fat intake in the adjusted meal is defined as the lower limit of the tentative dietary goal for preventing life-style related diseases for fats of users belonging to a category of “reference amount of energy.”

In the examples shown in FIGS. 1A and 1B, the upper limit and the lower limit of fat intake in the adjusted meal are defined as the upper limit and the lower limit of the tentative dietary goal for preventing life-style related diseases for fats in the category with the lowest estimated energy requirement, i.e., the category of female, 50 to 64 years of age, and physical activity level I (low). In this case, the upper limit of fat intake in the adjusted meal is 55.0 g/day (=1650 kcal/9 kcal×30%), and the lower limit of fat intake is 36.7 g/day (=1650 kcal/9 kcal×20%) since 1 g of fat provides 9 kcal of energy in the body.

Further, as another adjusting method to ensure intake of each component, it is more preferable that the values obtained by defining the above-mentioned number of calories taken in a day as 1650 kcal in the category with the lowest estimated energy requirement, i.e., the category of female, 50 to 64 years of age, and physical activity level I (low), in the Dietary Reference Intakes for Japanese for 18 to 64 years of age shown in FIGS. 1A and 1B and dividing the number of calories taken in a meal with adjusted nutritional components to be eaten (breakfast, lunch, or evening meal) by the above-mentioned 1650 kcal are within a range between the above-mentioned upper limit and lower limit of fat intake (55.0 g/day and 36.7 g/day).

Further, it is preferable not to take an excessive amount of saturated fatty acids, which are classified into a type of fats.

Saturated fatty acid intake is preferably determined within a range between the values obtained by preferably setting the upper limit of daily saturated fatty acid intake as described below and multiplying this upper limit by the value obtained by dividing the number of calories taken in a meal to be eaten (breakfast, lunch, or evening meal) by the total number of calories designed to be taken in a day (i.e., percentage of calorie intake of each meal).

In the “Dietary Reference Intakes for Japanese,” 7% of energy per day is established as the Dietary Reference Intakes (a tentative dietary goal for preventing life-style related diseases) of saturated fatty acids for men and women aged 18 years or older. Since the estimated energy requirement varies for each category of sex, age, and physical activity level, the upper limit and the lower limit vary depending on the category when the upper limit of saturated fatty acid is calculated in g/day as shown in FIGS. 1A and 1B.

Accordingly, the upper limit of saturated fatty acid intake in the adjusted meal is defined as the upper limit of the tentative dietary goal for preventing life-style related diseases for saturated fatty acids in the “reference amount of energy” category with the lowest estimated energy requirement, so that users with different sex, age, and physical activity level can eat a nutritionally complete diet that meets the Dietary Reference Intakes for Japanese.

In the examples shown in FIGS. 1A and 1B, the upper limit of saturated fatty acid intake in the adjusted meal is defined as the upper limit of the tentative dietary goal for preventing life-style related diseases for saturated fatty acids in the category with the lowest estimated energy requirement, i.e., the category of female, 50 to 64 years of age, and physical activity level I (low). In this case, the upper limit of saturated fatty acid intake in the adjusted meal is 12.8 g/day (=1650/9×0.07) since 1 g of saturated fatty acid provides 9 kcal of energy in the body.

Further, as another adjusting method, saturated fatty acid intake can also be determined within a range not more than the value obtained by defining the above-mentioned number of calories taken as 1650 kcal in the category with the lowest estimated energy requirement, i.e., the category of female, 50 to 64 years of age, and physical activity level I (low), in the Dietary Reference Intakes for Japanese for 18 to 64 years of age shown in FIGS. 1A and 1B and multiplying the above-mentioned upper limit of saturated fatty acid intake (12.8 g/day) by the value obtained by dividing the number of calories taken in a meal with adjusted nutritional components to be eaten (breakfast, lunch, or evening meal) by the above-mentioned 1650 kcal.

Further, it is preferable to take n-3 fatty acids and n-6 fatty acids, which are classified into types of fats, as described below.

Intakes of n-3 fatty acids and n-6 fatty acids are preferably within a range not less than the values obtained by preferably setting the lower limits of daily intakes of n-3 fatty acids and n-6 fatty acids as described below and multiplying these lower limits by the value obtained by dividing the number of calories taken in a meal to be eaten (breakfast, lunch, or evening meal) by the total number of calories designed to be taken in a day (i.e., percentage of calorie intake of each meal).

In the “Dietary Reference Intakes for Japanese,” the Dietary Reference Intakes of n-3 fatty acids and n-6 fatty acids are established as adequate intakes. As the Dietary Reference Intakes of n-3 fatty acids, 2.0 g/day and 1.6 g/day are established for men and women, respectively, aged 18 to 29 years, 2.0 g/day and 1.6 g/day are established for men and women, respectively, aged 30 to 49 years, and 2.2 g/day and 1.9 g/day are established for men and women, respectively, aged 50 to 64 years. As the Dietary Reference Intakes of n-6 fatty acids, 11 g/day and 8 g/day are established for men and women, respectively, aged 18 to 29 years, 10 g/day and 8 g/day are established for men and women, respectively, aged 30 to 49 years, and 10 g/day and 8 g/day are established for men and women, respectively, aged 50 to 64 years.

In this embodiment, the lower limits of intakes of n-3 fatty acids and n-6 fatty acids in the adjusted meal of the embodiment are defined as the maximum values (2.2 g/day for n-3 fatty acids and 11 g/day for n-6 fatty acids) of the adequate intakes among all categories, so that all users can take the adequate intakes of n-3 fatty acids and n-6 fatty acids by taking the adjusted meal.

Further, as another adjusting method, it is also preferable that intakes of n-3 fatty acids and n-6 fatty acids are determined within a range between the values obtained by defining the above-mentioned number of calories taken as 1650 kcal in the category with the lowest estimated energy requirement, i.e., the category of female, 50 to 64 years of age, and physical activity level I (low), in the Dietary Reference Intakes for Japanese for 18 to 64 years of age shown in FIGS. 1A and 1B and multiplying the above-mentioned lower limits of intakes of n-3 fatty acids and n-6 fatty acids by the value obtained by dividing the number of calories taken in a meal with adjusted nutritional components to be eaten (breakfast, lunch, or evening meal) by the above-mentioned 1650 kcal.

In other words, since the lower limit of n-3 fatty acids is 2.2 g/day, and that of n-6 fatty acid is 11 g/day, it is also preferable to define the lower limits as the values obtained by multiplying these values.

### —Carbohydrates (Including Dietary Fiber)—

Carbohydrates and dietary fiber contained in carbohydrates are taken in consideration of the PFC balance, and intakes of carbohydrates and dietary fiber contained in carbohydrates are more preferably determined within a range between the values obtained by preferably setting the upper limit and the lower limit of daily carbohydrate intake as described below and multiplying these upper limit and lower limit by the value obtained by dividing the number of calories taken in a meal to be eaten (breakfast, lunch, or evening meal) by the total number of calories designed to be taken in a day (i.e., percentage of calorie intake of each meal).

That is, the upper limit and the lower limit of intakes of carbohydrates and dietary fiber contained in carbohydrates are calculated as described below.

In the “Dietary Reference Intakes for Japanese,” 50% to 65% of energy per day is established as the Dietary Reference Intakes (a tentative dietary goal for preventing life-style related diseases) of carbohydrates for men and women aged 18 to 64 years. Since the estimated energy requirement varies for each category of sex, age, and physical activity level, the upper limit and the lower limit of the tentative dietary goal for preventing life-style related diseases for carbohydrates vary depending on the category as shown in FIGS. 1A and 1B.

Accordingly, the upper limit of carbohydrate intake in the adjusted meal is defined as the upper limit of the tentative dietary goal for preventing life-style related diseases for carbohydrates in the “reference amount of energy” category with the lowest estimated energy requirement, so that users with different sex, age, and physical activity level can eat a nutritionally complete diet that meets the Dietary Reference Intakes for Japanese.

Meanwhile, since the “recommended dietary allowance” is not established for carbohydrates, the lower limit of carbohydrate intake in the adjusted meal is defined as the lower limit of the tentative dietary goal for preventing life-style related diseases for carbohydrates of users belonging to the category of “reference amount of energy.”

In the example of FIGS. 1A and 1B, the upper limit and the lower limit of carbohydrate intake in the adjusted meal are the upper limit and the lower limit of the tentative dietary goal for preventing life-style related diseases for carbohydrates for users belonging to the category with the lowest estimated energy requirement, i.e., the category of female, 50 to 64 years of age, and physical activity level I (low). In this case, the upper limit of carbohydrate intake is 268.1 g/day (=1650/4×0.65), and the lower limit of carbohydrate intake is 206.3 g/day (=1650/4×0.5) since 1 g of carbohydrate provides 4 kcal of energy.

Further, as another adjusting method, it is also preferable that carbohydrate intake is determined within a range between the values obtained by defining the total number of calories taken in a day as 1650 kcal in the category with the lowest estimated energy requirement, i.e., the category of female, 50 to 64 years of age, and physical activity level I (low), in the Dietary Reference Intakes for Japanese for 18 to 64 years of age shown in FIGS. 1A and 1B and multiplying the above-mentioned upper limit and lower limit of carbohydrate intake (268.1 g/day and 206.3 g/day) by the value obtained by dividing the number of calories taken in a meal with adjusted nutritional components to be eaten (breakfast, lunch, or evening meal) by the above-mentioned 1650 kcal.

Further, as described below, it is also preferable that dietary fiber intake is determined within a range not less than the value obtained by multiplying the lower limit of daily dietary fiber intake by the value obtained by dividing the number of calories taken in a meal to be eaten (breakfast, lunch, or evening meal) by the total number of calories designed to be taken in a day (i.e., percentage of calorie intake of each meal).

Further, in the “Dietary Reference Intakes for Japanese,” the Dietary Reference Intakes of dietary fiber are established as the tentative dietary goal for preventing life-style related diseases, and 21 g/day or more and 18 g/day or more for men and women, respectively, aged 18 to 64 years of age are established as the tentative dietary goals for preventing life-style related diseases. In this embodiment, the lower limit of dietary fiber intake in the adjusted meal of this embodiment is defined as the maximum value (21 g/day) of the tentative dietary goal for preventing life-style related diseases for dietary fiber among all categories, so that all users can achieve the tentative dietary goal for preventing life-style related diseases for dietary fiber by eating the adjusted meal with an adjusted amount.

Further, as another adjusting method, it is also preferable that dietary fiber intake is determined within a range not less than the value obtained by defining the total number of calories taken in a day as 1650 kcal in the category with the lowest estimated energy requirement, i.e., the category of female, 50 to 64 years of age, and physical activity level I (low), in the Dietary Reference Intakes for Japanese for 18 to 64 years of age shown in FIGS. 1A and 1B and multiplying the above-mentioned lower limit of dietary fiber intake (21 g/day) by the value obtained by dividing the number of calories taken in a meal with adjusted nutritional components to be eaten (breakfast, lunch, or evening meal) by the above-mentioned 1650 kcal.

### —Vitamins, Minerals—

In the present invention, the amounts of contained vitamins and minerals need to be adjusted in the meal with adjusted nutritional components. Preferred embodiments of each component are described below. It is sufficient to adjust at least one component among these, and it is preferable that the nutrients to be adjusted include at least “vitamin B1 and vitamin C” and “Ca, Mg, and Fe,” which are the vitamins and minerals shown to be inadequately taken by 20% or more on the basis of the Dietary Reference Intakes in a public health/nutrition survey.

It is more preferable that the nutrients to be adjusted include at least “vitamin A, vitamin B1, vitamin B2, vitamin B6, folic acid, and vitamin C” and “K, Ca, Mg, P, and Fe,” which are the vitamins and minerals shown to be inadequately taken on the basis of the Dietary Reference Intakes in a public health/nutrition survey.

It is most preferable that the nutrients to be adjusted include all the following vitamins and minerals.

### Sodium (Salt Equivalent)

It is recommended to restrict the amount of sodium in the adjusted meal of the present invention, and sodium intake is preferably determined within a range between the values obtained by preferably setting the upper limit and the lower limit of daily sodium (salt equivalent) intake as described below and multiplying these upper limit and lower limit by the value obtained by dividing the number of calories taken in a meal to be eaten (breakfast, lunch, or evening meal) by the total number of calories designed to be taken in a day (i.e., percentage of calorie intake of each meal).

That is, the upper limit and the lower limit sodium intake are calculated as described below. In the “Dietary Reference Intakes for Japanese,” less than 7.5 g/day for men and less than 6.5 g/day for women in terms of salt equivalent are established as the Dietary Reference Intakes (a tentative dietary goal for preventing life-style related diseases) of sodium for men and women aged 18 to 64 years.

Here, the amount of the adjusted meal of this embodiment is adjusted depending on the attributes of each user. Therefore, the salt equivalent contained in the meal with an adjusted amount needs to be lower than the Dietary Reference Intakes in the category with high estimated energy requirement. Accordingly, a normalized salt equivalent in each category is calculated by the following equation, and the upper limit of the salt equivalent of sodium intake in the adjusted meal is defined as the lowest normalized salt equivalent among all.

Normalized salt equivalent=tentative dietary goal for preventing life-style related diseases for salt equivalents×(reference amount of energy/estimated energy requirement in each category)

In the example of FIGS. 1A and 1B, since the estimated energy requirement in the category of male, 18 to 29 years of age, and physical activity level III (high) is higher than in other categories, the salt equivalent that can be added to the adjusted meal is 4.06 g/day (7.5 g×1650 kcal/3050 kcal), which is the lowest value. Accordingly, sodium in the adjusted meal is preferably less than 4.06 g/day, which is the upper limit of sodium intake, in terms of salt equivalent.

Meanwhile, since sodium influences the taste of a meal, the amount can be selected flexibly. For example, the amount of sodium (salt equivalent) is classified into less than 3.0 g as “proper” and less than 3.5 g as “strong” according to the Smart Meal Criteria, which were reviewed and approved by a consortium comprising The Japanese Society of Nutrition and Dietetics, Japanese Society of Nutrition and Foodservice Management, Japanese Society of Hypertension, and others, and these criteria may be followed. In other words, a method for adjusting the salt equivalent to less than the upper limit can be implemented by setting the salt equivalent at less than 3.0 g if the energy intake in daily breakfast, lunch, or evening meal is 450 to 650 kcal or at less than 3.5 g if the energy intake is 650 to 850 kcal. Further, other independent criteria may be established. For example, if the energy intake is less than 450 kcal, the salt equivalent may be less than 2.5 g. If the energy intake is 850 kcal or higher, it may be less than 4.0 g.

Further, as another independent criteria, a method for adjusting the salt equivalent to the upper limit can be implemented by setting the salt intake at 1.7 g or less for breakfast, less than 3.0 g for lunch, and less than 3.5 g for evening meal among daily breakfast, lunch, and evening meal. The upper limit of sodium intake (salt equivalent) can also be set in this way.

Further, criteria other than the above-described setting methods may be followed, or criteria for eating delicious food and reducing the salt consumption without difficulty may be established.

Calcium

It is recommended to take calcium in consideration of balance, and calcium intake is more preferably determined within a range between the values obtained by preferably setting the upper limit and the lower limit of daily calcium intake as described below and multiplying these upper limit and lower limit by the value obtained by dividing the number of calories taken in an adjusted meal to be eaten (breakfast, lunch, or evening meal) by the total number of calories designed to be taken in a day (i.e., percentage of calorie intake of each meal).

Here, the upper limit and the lower limit of daily calcium intake are preferably calculated as described below. In the “Dietary Reference Intakes for Japanese,” the recommended dietary allowance and the tolerable upper intake level of calcium are established as the Dietary Reference Intakes for 18 to 64 years of age.

Here, the amount of the adjusted meal of this embodiment is adjusted depending on the attributes of each user. Therefore, calcium contained in the adjusted meal with an adjusted amount needs to be less than the Dietary Reference Intakes in the category with high estimated energy requirement. Accordingly, a normalized calcium amount is calculated by the following equation for each category, and the upper limit of calcium intake in the adjusted meal is defined as the least normalized calcium amount among all.

Normalized calcium amount=tolerable upper intake level of calcium×(reference amount of energy/estimated energy requirement for each category)

Further, the lower limit of calcium intake in the reference adjusted meal is defined as the maximum value (800 mg/day) of the recommended dietary allowance of calcium among all categories, so that all users can take calcium not less than the recommended dietary allowance.

In the example in FIGS. 1A and 1B, since the estimated energy requirement in the category of male, 18 to 29 years of age, and physical activity level III (high) is higher than in other categories, calcium that can be contained in the adjusted meal is 1352.5 mg/day (=2500 mg×1650 kcal/3050 kcal), which is the lowest value. Accordingly, this value is specified as the upper limit of calcium intake in the adjusted meal.

Further, as another adjusting method, it is also preferable that calcium intake is determined within a range between the values obtained by defining the total number of calories taken in a day as 1650 kcal in the category with the lowest estimated energy requirement, i.e., the category of female, 50 to 64 years of age, and physical activity level I (low), in the Dietary Reference Intakes for Japanese for 18 to 64 years of age shown in FIGS. 1A and 1B and multiplying the above-mentioned upper limit and lower limit of calcium intake (1352.5 mg/day and 800 mg/day) by the value obtained by dividing the number of calories taken in a meal with adjusted nutritional components to be eaten (breakfast, lunch, or evening meal) by the above-mentioned 1650 kcal.

Iron

It is recommended to take iron in consideration of balance, and iron intake is more preferably determined within a range between the values obtained by preferably setting the upper limit and the lower limit of daily iron intake as described below and multiplying these upper limit and lower limit by the value obtained by dividing the number of calories taken in an adjusted meal to be eaten (breakfast, lunch, or evening meal) by the total number of calories designed to be taken in a day (i.e., percentage of calorie intake of each meal).

Here, the upper limit and lower limit of daily iron intake are preferably calculated as described below.

The upper limit and the lower limit of iron intake in the adjusted meal of this embodiment are calculated as described below.

In the “Dietary Reference Intakes for Japanese,” the recommended dietary allowance and the tolerable upper intake level are established as the Dietary Reference Intakes of iron for 18 to 64 years of age.

Here, the amount of the adjusted meal of this embodiment is adjusted depending on the attributes of each user. Therefore, iron contained in the adjusted meal with an adjusted amount needs to be less than the Dietary Reference Intakes in the category with high estimated energy requirement. Accordingly, a normalized iron amount is calculated by the following equation for each category, and the upper limit of iron intake in the adjusted meal is defined as the least normalized iron amount among all.

Normalized iron amount=tolerable upper intake level of iron×(reference amount of energy/estimated energy requirement for each category)

Further, the lower limit of iron intake in the adjusted meal is defined as the maximum value (10.5 mg/day) of the recommended dietary allowance of iron among all categories, so that all users can take iron not less than the recommended dietary allowance. In the example of FIGS. 1A and 1B, since the estimated energy requirement in the category of male, 18 to 29 years of age, and physical activity level III (high) is higher than in other categories, the amount of iron that can be contained in the adjusted meal is 27.0 mg/day (=50 mg×1650 kcal/3050 kcal), which is the lowest value. Accordingly, this value is specified as the upper limit of iron intake in the adjusted meal.

Further, as another adjusting method, it is also preferable that iron intake is determined within a range between the values obtained by defining the total number of calories taken in a day as 1650 kcal in the category with the lowest estimated energy requirement, i.e., the category of female, 50 to 64 years of age, and physical activity level I (low), in the Dietary Reference Intakes for Japanese for 18 to 64 years of age shown in FIGS. 1A and 1B and multiplying the above-mentioned upper limit and lower limit of iron intake (27.0 mg/day and 10.5 mg/day) by the value obtained by dividing the number of calories taken in a meal with adjusted nutritional components to be eaten (breakfast, lunch, or evening meal) by the above-mentioned 1650 kcal.

Phosphorus

It is recommended to take phosphorus in consideration of balance, and phosphorus intake is more preferably determined within a range between the values obtained by preferably setting the upper limit and the lower limit of daily phosphorus intake as described below and multiplying these upper limit and lower limit by the value obtained by dividing the number of calories taken in an adjusted meal to be eaten (breakfast, lunch, or evening meal) by the total number of calories designed to be taken in a day (i.e., percentage of calorie intake of each meal).

Here, the upper limit and lower limit of daily phosphorus intake are calculated as described below.

In the “Dietary Reference Intakes for Japanese,” the adequate intake and tolerable upper intake level of phosphorus are established as Dietary Reference Intakes for 18 to 64 years of age. Here, the amount of the adjusted meal of this embodiment is adjusted depending on the attributes of each user. Therefore, phosphorus contained in the adjusted meal with an adjusted amount needs to be less than the Dietary Reference Intakes for a category with high estimated energy requirement. Accordingly, a normalized phosphorus amount for each category is calculated by the following equation, and the upper limit of phosphorus intake in the adjusted meal is defined as the smallest normalized phosphorus amount among all.

Normalized phosphorus amount=tolerable upper intake level of phosphorus×(reference amount of energy/estimated energy requirement for each category)

Further, the lower limit of phosphorus intake in the adjusted meal is defined as the maximum value (1000 mg/day) of the adequate intake of phosphorus among all categories, so that all users can take phosphorus not less than the adequate intake. In the example of FIGS. 1A and 1B, since the estimated energy requirement in the category of male, 18 to 29 years of age, and physical activity level III (high) is higher than in other categories, phosphorus that can be contained in the adjusted meal is 1623.0 mg/day (=3000 mg×1650 kcal/3050 kcal), which is the lowest value. Accordingly, this value is specified as the upper limit of phosphorus intake in the adjusted meal.

Further, as another adjusting method, it is also preferable that phosphorus intake is determined within a range between the values obtained by defining the total number of calories taken in a day as 1650 kcal in the category with the lowest estimated energy requirement, i.e., the category of female, 50 to 64 years of age, and physical activity level I (low), in the Dietary Reference Intakes for Japanese for 18 to 64 years of age shown in FIGS. 1A and 1B and multiplying the above-mentioned upper limit and lower limit of phosphorus intake (1000 mg/day and 1623.0 mg/day) by the value obtained by dividing the number of calories taken in a meal with adjusted nutritional components to be eaten (breakfast, lunch, or evening meal) by the above-mentioned 1650 kcal.

Magnesium

It is recommended to take magnesium in consideration of balance, and magnesium intake is more preferably determined within a range not less than the value obtained by setting the lower limit of daily magnesium intake as described below and multiplying the lower limit by the value obtained by dividing the number of calories taken in an adjusted meal to be eaten (breakfast, lunch, or evening meal) by the total number of calories designed to be taken in a day (i.e., percentage of calorie intake of each meal).

Here, the lower limit of daily magnesium intake is calculated as described below. In the “Dietary Reference Intakes for Japanese,” the recommended dietary allowance in each category is established as the Dietary Reference Intakes of magnesium for 18 to 64 years of age.

Here, the lower limit of magnesium intake in the adjusted meal is defined as the maximum value (370 mg/day) of the recommended dietary allowance of magnesium among all categories, so that all users can take magnesium not less than the recommended dietary allowance.

Further, as another adjusting method, it is also preferable that magnesium intake is determined within a range not less than the value obtained by defining the total number of calories taken in a day as 1650 kcal in the category with the lowest estimated energy requirement, i.e., the category of female, 50 to 64 years of age, and physical activity level I (low), in the Dietary Reference Intakes for Japanese for 18 to 64 years of age shown in FIGS. 1A and 1B and multiplying the above-mentioned lower limit of magnesium intake (370 mg/day) by the value obtained by dividing the number of calories taken in a meal with adjusted nutritional components to be eaten (breakfast, lunch, or evening meal) by the above-mentioned 1650 kcal.

Potassium

It is recommended to take potassium in consideration of balance, and potassium intake is more preferably determined within a range not less than the value obtained by preferably setting the lower limit of daily potassium intake as described below and multiplying the lower limit by the value obtained by dividing the number of calories taken in an adjusted meal to be eaten (breakfast, lunch, or evening meal) by the total number of calories designed to be taken in a day (i.e., percentage of calorie intake of each meal).

The lower limit of potassium intake is calculated as described below. In the “Dietary Reference Intakes for Japanese,” the adequate intake is established as the Dietary Reference Intakes of potassium for 18 to 64 years of age. In this embodiment, the lower limit of potassium intake in the adjusted meal is defined as the maximum value (2500 mg/day) of the adequate intake of potassium among all categories, so that all users can take potassium not less than the adequate intake.

Further, as another adjusting method, it is also preferable that potassium intake is determined within a range not less than the value obtained by defining the total number of calories taken in a day as 1650 kcal in the category with the lowest estimated energy requirement, i.e., the category of female, 50 to 64 years of age, and physical activity level I (low), in the Dietary Reference Intakes for Japanese for 18 to 64 years of age shown in FIGS. 1A and 1B and multiplying the above-mentioned lower limit of potassium intake (2500 mg/day) by the value obtained by dividing the number of calories taken in a meal with adjusted nutritional components to be eaten (breakfast, lunch, or evening meal) by the above-mentioned 1650 kcal.

Copper

It is recommended to take copper in consideration of balance, and copper intake is more preferably determined within a range between the values obtained by preferably setting the upper limit and the lower limit of daily copper intake as described below and multiplying these upper limit and lower limit by the value obtained by dividing the number of calories taken in an adjusted meal to be eaten (breakfast, lunch, or evening meal) by the total number of calories designed to be taken in a day (i.e., percentage of calorie intake of each meal).

The upper limit and the lower limit of copper intake are calculated as described below. In the “Dietary Reference Intakes for Japanese,” the recommended dietary allowance and the tolerable upper intake level are established as the Dietary Reference Intakes of copper for 18 to 64 years of age.

Here, the amount of the adjusted meal of this embodiment is adjusted depending on the attributes of each user. Therefore, copper contained in the adjusted meal with an adjusted amount needs to be less than the Dietary Reference Intakes for a category with high estimated energy requirement. Accordingly, a normalized copper amount in each category is calculated by the following equation, and the upper limit of copper intake in the adjusted meal is defined as the smallest normalized copper amount among all.

Normalized copper amount=tolerable upper intake level of copper×(reference amount of energy/estimated energy requirement for each category)

Further, the lower limit of copper intake in the adjusted meal is defined as the maximum value (0.9 mg/day) of the recommended dietary allowance of copper among all categories, so that all users can take copper not less than the recommended dietary allowance.

In the example of FIGS. 1A and 1B, since the estimated energy requirement in the category of male, 18 to 29 years of age, and physical activity level III (high) is higher than in other categories, the amount of copper that can be contained in the adjusted meal is 3.79 mg/day (7 mg×1650 kcal/3050 kcal), which is the lowest value. Accordingly, this value is specified as the upper limit of copper intake in the adjusted meal.

Further, as another adjusting method, it is also preferable that copper intake is determined within a range between the values obtained by defining the total number of calories taken in a day as 1650 kcal in the category with the lowest estimated energy requirement, i.e., the category of female, 50 to 64 years of age, and physical activity level I (low), in the Dietary Reference Intakes for Japanese for 18 to 64 years of age shown in FIGS. 1A and 1B and multiplying the above-mentioned upper limit and lower limit of copper intake (0.9 mg/day and 3.79 mg/day) by the value obtained by dividing the number of calories taken in a meal with adjusted nutritional components to be eaten (breakfast, lunch, or evening meal) by the above-mentioned 1650 kcal.

Iodine

It is recommended to take iodine in consideration of balance, and iodine intake is preferably determined within a range between the values obtained by preferably setting the upper limit and the lower limit of daily iodine intake as described below and multiplying these upper limit and lower limit by the value obtained by dividing the number of calories taken in an adjusted meal to be eaten (breakfast, lunch, or evening meal) by the total number of calories designed to be taken in a day (i.e., percentage of calorie intake of each meal).

The upper limit and the lower limit of iodine intake are calculated as described below. In the “Dietary Reference Intakes for Japanese,” the recommended dietary allowance and the tolerable upper intake level are established as the Dietary Reference Intakes of iodine for 18 to 64 years of age.

Here, the amount of the adjusted meal of this embodiment is adjusted depending on the attributes of each user. Therefore, iodine contained in the adjusted meal with an adjusted amount needs to be less than the Dietary Reference Intakes for a category with high estimated energy requirement. Accordingly, a normalized iodine amount in each category is calculated by the following equation, and the upper limit of iodine intake in the adjusted meal is defined as the smallest normalized iodine amount among all.

Normalized iodine amount=tolerable upper intake level of iodine×(reference amount of energy/estimated energy requirement for each category)

Further, the lower limit of iodine intake in the adjusted meal is defined as the maximum value (130 μg/day) of the recommended dietary allowance of iodine among all categories, so that all users can take iodine not less than the recommended dietary allowance.

In the example of FIGS. 1A and 1B, since the estimated energy requirement in the category of male, 18 to 29 years of age, and physical activity level III (high) is higher than in other categories, the amount of iodine that can be contained in the adjusted meal is 1623 μg/day (3000 μg×1650 kcal/3050 kcal), which is the lowest value. Accordingly, this value is specified as the upper limit of iodine intake in the adjusted meal.

Of note, the tolerable upper intake level in the “Dietary Reference Intakes for Japanese” is established for habitual intake, and the tolerable upper intake level is allowed to exceed intermittently, which is also applicable in this embodiment.

Further, as another adjusting method, it is also preferable that iodine intake is determined within a range between the values obtained by defining the total number of calories taken in a day as 1650 kcal in the category with the lowest estimated energy requirement, i.e., the category of female, 50 to 64 years of age, and physical activity level I (low), in the Dietary Reference Intakes for Japanese for 18 to 64 years of age shown in FIGS. 1A and 1B and multiplying the above-mentioned upper limit and lower limit of iodine intake (130 μg/day and 1623 μg/day) by the value obtained by dividing the number of calories taken in a meal with adjusted nutritional components to be eaten (breakfast, lunch, or evening meal) by the above-mentioned 1650 kcal.

Selenium

It is recommended to take selenium in consideration of balance, and selenium intake is preferably determined within a range between the values obtained by preferably setting the upper limit and the lower limit of daily selenium intake as described below and multiplying these upper limit and lower limit by the value obtained by dividing the number of calories taken in an adjusted meal to be eaten (breakfast, lunch, or evening meal) by the total number of calories designed to be taken in a day (i.e., percentage of calorie intake of each meal).

The upper limit and the lower limit of selenium intake are calculated as described below. In the “Dietary Reference Intakes for Japanese,” the recommended dietary allowance and the tolerable upper intake level are established as the Dietary Reference Intakes of selenium for 18 to 64 years of age.

Here, the amount of the adjusted meal of this embodiment is adjusted depending on the attributes of each user. Therefore, the amount of selenium contained in the adjusted meal with an adjusted amount needs to be less than the Dietary Reference Intakes for a category with high estimated energy requirement. Accordingly, a normalized selenium amount in each category is calculated by the following equation, and the upper limit of selenium intake in the adjusted meal is defined as the smallest normalized selenium amount among all.

Normalized selenium amount=tolerable upper intake level of selenium×(reference amount of energy/estimated energy requirement for each category)

Further, the lower limit of selenium intake in the adjusted meal is defined as the maximum value (30 μg/day) of the recommended dietary allowance of selenium among all categories, so that all users can take selenium not less than the recommended dietary allowance.

In the example of FIGS. 1A and 1B, since the estimated energy requirement in the category of male, 18 to 29 years of age, and physical activity level III (high) is higher than in other categories, the amount of selenium that can be contained in the adjusted meal is 243.4 μg/day (450 μg×1650 kcal/3050 kcal), which is the lowest value. Accordingly, this value is specified as the upper limit of selenium intake in the adjusted meal.

Further, as another adjusting method, it is also preferable that selenium intake is determined within a range between the values obtained by defining the total number of calories taken in a day as 1650 kcal in the category with the lowest estimated energy requirement, i.e., the category of female, 50 to 64 years of age, and physical activity level I (low), in the Dietary Reference Intakes for Japanese for 18 to 64 years of age shown in FIGS. 1A and 1B and multiplying the above-mentioned upper limit and lower limit of selenium intake (30 μg/day and 243.4 μg/day) by the value obtained by dividing the number of calories taken in a meal with adjusted nutritional components to be eaten (breakfast, lunch, or evening meal) by the above-mentioned 1650 kcal.

Zinc

It is recommended to take zinc in consideration of balance, and zinc intake is preferably determined within a range between the values obtained by preferably setting the upper limit and the lower limit of daily zinc intake as described below and multiplying these upper limit and lower limit by the value obtained by dividing the number of calories taken in an adjusted meal to be eaten (breakfast, lunch, or evening meal) by the total number of calories designed to be taken in a day (i.e., percentage of calorie intake of each meal).

Here, the upper limit and the lower limit of zinc intake are calculated as described below. In the “Dietary Reference Intakes for Japanese,” the recommended dietary allowance and the tolerable upper intake level are established as the Dietary Reference Intakes of zinc for 18 to 64 years of age.

Here, the amount of the adjusted meal of this embodiment is adjusted depending on the attributes of each user. Therefore, the amount of zinc contained in the adjusted meal with an adjusted amount needs to be less than the Dietary Reference Intakes for a category with high estimated energy requirement. Accordingly, a normalized zinc amount in each category is calculated by the following equation, and the upper limit of zinc intake in the adjusted meal is defined as the smallest normalized zinc amount among all.

Normalized zinc amount=tolerable upper intake level of zinc×(reference amount of energy/estimated energy requirement for each category)

Further, the lower limit of zinc intake in the adjusted meal is defined as the maximum value (11 mg/day) of the recommended dietary allowance of zinc among all categories, so that all users can take zinc not less than the recommended dietary allowance.

In the example of FIGS. 1A and 1B, since the estimated energy requirement in the category of male, 18 to 29 years of age, and physical activity level III (high) is higher than in other categories, the amount of zinc that can be contained in the adjusted meal is 21.6 mg/day (40 mg×1650 kcal/3050 kcal), which is the lowest value. Accordingly, this value is specified as the upper limit of zinc intake in the adjusted meal.

Further, as another adjusting method, it is also preferable that zinc intake is determined within a range between the values obtained by defining the total number of calories taken in a day as 1650 kcal in the category with the lowest estimated energy requirement, i.e., the category of female, 50 to 64 years of age, and physical activity level I (low), in the Dietary Reference Intakes for Japanese for 18 to 64 years of age shown in FIGS. 1A and 1B and multiplying the above-mentioned upper limit and lower limit of zinc intake (11 mg/day and 21.6 mg/day) by the value obtained by dividing the number of calories taken in a meal with adjusted nutritional components to be eaten (breakfast, lunch, or evening meal) by the above-mentioned 1650 kcal.

Chromium

It is recommended to take chromium in consideration of balance, and chromium intake is preferably determined within a range between the values obtained by preferably setting the upper limit and the lower limit of daily chromium intake as described below and multiplying these upper limit and lower limit by the value obtained by dividing the number of calories taken in a meal with adjusted nutritional components to be eaten (breakfast, lunch, or evening meal) by the total number of calories designed to be taken in a day (i.e., percentage of calorie intake of each meal).

The upper limit and the lower limit of chromium intake are calculated as described below.

In the “Dietary Reference Intakes for Japanese,” the adequate intake and the tolerable upper intake level are established as the Dietary Reference Intakes of chromium for 18 to 64 years of age. Here, the amount of the adjusted meal of this embodiment is adjusted depending on the attributes of each user. Therefore, the amount of chromium contained in the adjusted meal with an adjusted amount needs to be less than the Dietary Reference Intakes for a category with high estimated energy requirement. Accordingly, a normalized chromium amount in each category is calculated by the following equation, and the upper limit of chromium intake in the adjusted meal is defined as the smallest normalized chromium amount among all.

Normalized chromium amount=tolerable upper intake level of chromium×(reference amount of energy/estimated energy requirement for each category)

Further, the lower limit of chromium intake in the adjusted meal is defined as the maximum value (10 μg/day) of the adequate intake of chromium among all categories, so that all users can take chromium not less than the adequate intake. In the example of FIGS. 1A and 1B, since the estimated energy requirement in the category of male, 18 to 29 years of age, and physical activity level III (high) is higher than in other categories, the amount of chromium that can be contained in the adjusted meal is 270.5 μg/day (500 μg×1650 kcal/3050 kcal), which is the lowest value. Accordingly, this value is specified as the upper limit of chromium intake in the adjusted meal.

Further, as another adjusting method, it is also preferable that chromium intake is determined within a range between the values obtained by defining the total number of calories taken in a day as 1650 kcal in the category with the lowest estimated energy requirement, i.e., the category of female, 50 to 64 years of age, and physical activity level I (low), in the Dietary Reference Intakes for Japanese for 18 to 64 years of age shown in FIGS. 1A and 1B and multiplying the above-mentioned upper limit and lower limit of chromium intake (10 μg/day and 270.5 μg/day) by the value obtained by dividing the number of calories taken in a meal with adjusted nutritional components to be eaten (breakfast, lunch, or evening meal) by the above-mentioned 1650 kcal.

Manganese

It is recommended to take manganese in consideration of balance, and manganese intake is preferably determined within a range between the values obtained by preferably setting the upper limit and the lower limit of daily manganese intake as described below and multiplying these upper limit and lower limit by the value obtained by dividing the number of calories taken in an adjusted meal to be eaten (breakfast, lunch, or evening meal) by the total number of calories designed to be taken in a day (i.e., percentage of calorie intake of each meal).

Here, the upper limit and the lower limit of manganese intake are calculated as described below. In the “Dietary Reference Intakes for Japanese,” the adequate intake and the tolerable upper intake level are established as the Dietary Reference Intakes of manganese for 18 to 64 years of age.

Here, the amount of the adjusted meal of this embodiment is adjusted depending on the attributes of each user. Therefore, the amount of manganese contained in the adjusted meal with an adjusted amount needs to be less than the Dietary Reference Intakes for a category with high estimated energy requirement. Accordingly, a normalized manganese amount in each category is calculated by the following equation, and the upper limit of manganese intake in the adjusted meal is defined as the smallest normalized manganese amount among all.

Normalized manganese amount=tolerable upper intake level of manganese×(reference amount of energy/estimated energy requirement for each category)

Further, the lower limit of manganese intake in the adjusted meal is defined as the maximum value (4 mg/day) of the adequate intake of manganese among all categories, so that all users can take manganese not less than the adequate intake.

In the example of FIGS. 1A and 1B, since the estimated energy requirement in the category of male, 18 to 29 years of age, and physical activity level III (high) is higher than in other categories, the amount of manganese that can be contained in the adjusted meal is 6.0 mg/day (11 mg×1650 kcal/3050 kcal), which is the lowest value. Accordingly, this value is specified as the upper limit of manganese intake in the adjusted meal.

Further, as another adjusting method, it is also preferable that manganese intake is determined within a range between the values obtained by defining the total number of calories taken in a day as 1650 kcal in the category with the lowest estimated energy requirement, i.e., the category of female, 50 to 64 years of age, and physical activity level I (low), in the Dietary Reference Intakes for Japanese for 18 to 64 years of age shown in FIGS. 1A and 1B and multiplying the above-mentioned upper limit and lower limit of manganese intake (4 mg/day and 6.0 mg/day) by the value obtained by dividing the number of calories taken in a meal with adjusted nutritional components to be eaten (breakfast, lunch, or evening meal) by the above-mentioned 1650 kcal.

Molybdenum

It is recommended to take molybdenum in consideration of balance, and molybdenum intake is preferably determined within a range between the values obtained by preferably setting the upper limit and the lower limit of daily molybdenum intake as described below and multiplying these upper limit and lower limit by the value obtained by dividing the number of calories taken in an adjusted meal to be eaten (breakfast, lunch, or evening meal) by the total number of calories designed to be taken in a day (i.e., percentage of calorie intake of each meal).

Here, the upper limit and the lower limit of molybdenum intake are calculated as described below. In the “Dietary Reference Intakes for Japanese,” the recommended dietary allowance and the tolerable upper intake level are established as the Dietary Reference Intakes of molybdenum for 18 to 64 years of age.

Here, the amount of the adjusted meal of this embodiment is adjusted depending on the attributes of each user. Therefore, the amount of molybdenum contained in the adjusted meal with an adjusted amount needs to be less than the Dietary Reference Intakes for a category with high estimated energy requirement. Accordingly, a normalized molybdenum amount in each category is calculated by the following equation, and the upper limit of molybdenum intake in the adjusted meal is defined as the smallest normalized molybdenum amount among all.

Normalized molybdenum amount=tolerable upper intake level of molybdenum×(reference amount of energy/estimated energy requirement for each category)

Further, the lower limit of molybdenum intake in the adjusted meal is defined as the maximum value (30 μg/day) of the recommended dietary allowance of molybdenum among all categories, so that all users can take molybdenum not less than the recommended dietary allowance.

In the example of FIGS. 1A and 1B, since the estimated energy requirement in the category of male, 18 to 29 years of age, and physical activity level III (high) is higher than in other categories, the amount of molybdenum that can be contained in the adjusted meal is 324.6 μg/day (600 μg×1650 kcal/3050 kcal), which is the lowest value. Accordingly, this value is specified as the upper limit of molybdenum intake in the adjusted meal.

Further, as another adjusting method, it is also preferable that molybdenum intake is determined within a range between the values obtained by defining the total number of calories taken in a day as 1650 kcal in the category with the lowest estimated energy requirement, i.e., the category of female, 50 to 64 years of age, and physical activity level I (low), in the Dietary Reference Intakes for Japanese for 18 to 64 years of age shown in FIGS. 1A and 1B and multiplying the above-mentioned upper limit and lower limit of molybdenum intake (30 μg/day and 324.6 μg/day) by the value obtained by dividing the number of calories taken in a meal with adjusted nutritional components to be eaten (breakfast, lunch, or evening meal) by the above-mentioned 1650 kcal.

Vitamin A

It is recommended to take vitamin A in consideration of balance, and vitamin A intake is preferably determined within a range between the values obtained by preferably setting the upper limit and the lower limit of daily vitamin A intake as described below and multiplying these upper limit and lower limit by the value obtained by dividing the number of calories taken in an adjusted meal to be eaten (breakfast, lunch, or evening meal) by the total number of calories designed to be taken in a day (i.e., percentage of calorie intake of each meal).

Here, the upper limit and the lower limit of vitamin A intake are calculated as described below. In the “Dietary Reference Intakes for Japanese,” the recommended dietary allowance and the tolerable upper intake level are established as the Dietary Reference Intakes of vitamin A for 18 to 64 years of age.

The amount of the adjusted meal of this embodiment is adjusted depending on the attributes of each user. Therefore, the amount of vitamin A contained in the adjusted meal with an adjusted amount needs to be less than the Dietary Reference Intakes for a category with high estimated energy requirement. Accordingly, a normalized vitamin A amount in each category is calculated by the following equation, and the upper limit of vitamin A intake in the adjusted meal is defined as the smallest normalized vitamin A amount among all.

Normalized vitamin A amount=tolerable upper intake level of vitamin A×(reference amount of energy/estimated energy requirement for each category)

Further, the lower limit of vitamin A intake in the adjusted meal is defined as the maximum value (900 μg RE/day) of the recommended dietary allowance of vitamin A among all categories, so that all users can take vitamin A not less than the recommended dietary allowance.

In the example of FIGS. 1A and 1B, since the estimated energy requirement in the category of male, 18 to 29 years of age, and physical activity level III (high) is higher than in other categories, the amount of vitamin A that can be contained in the adjusted meal is 1460.7 μg RE/day (2700 μg RE×1650 kcal/3050 kcal), which is the lowest value. Accordingly, this value is specified as the upper limit of vitamin A intake in the adjusted meal.

Of note, vitamin A is classified into retinol (found in animal food) and carotenoid (found in plant food). In the “Dietary Reference Intakes for Japanese,” the upper limit is established only for retinol and vitamin A additives and is not established for meal-derived carotenoid, which is also applicable in this embodiment.

Further, as another adjusting method, it is also preferable that vitamin A intake is determined within a range between the values obtained by defining the total number of calories taken in a day as 1650 kcal in the category with the lowest estimated energy requirement, i.e., the category of female, 50 to 64 years of age, and physical activity level I (low), in the Dietary Reference Intakes for Japanese for 18 to 64 years of age shown in FIGS. 1A and 1B and multiplying the above-mentioned upper limit and lower limit of vitamin A intake (900 μg RE/day and 1460.7 μg RE/day) by the value obtained by dividing the number of calories taken in a meal with adjusted nutritional components to be eaten (breakfast, lunch, or evening meal) by the above-mentioned 1650 kcal.

Vitamin D

It is recommended to take vitamin D in consideration of balance, and vitamin D intake is preferably determined within a range between the values obtained by preferably setting the upper limit and the lower limit of daily vitamin D intake as described below and multiplying these upper limit and lower limit by the value obtained by dividing the number of calories taken in an adjusted meal to be eaten (breakfast, lunch, or evening meal) by the total number of calories designed to be taken in a day (i.e., percentage of calorie intake of each meal).

Here, the upper limit and the lower limit of vitamin D intake are calculated as described below.

In the “Dietary Reference Intakes for Japanese,” the adequate intake and the tolerable upper intake level are established as the Dietary Reference Intakes of vitamin D for 18 to 64 years of age. Here, the amount of the adjusted meal of this embodiment is adjusted depending on the attributes of each user. Therefore, the amount of vitamin D contained in the adjusted meal with an adjusted amount needs to be less than the Dietary Reference Intakes for a category with high estimated energy requirement. Accordingly, a normalized vitamin D amount in each category is calculated by the following equation, and the upper limit of vitamin D intake in the adjusted meal is defined as the smallest normalized vitamin D amount among all.

Normalized vitamin D amount=tolerable upper intake level of vitamin D×(reference amount of energy/estimated energy requirement for each category)

Further, the lower limit of vitamin D intake in the adjusted meal is defined as the maximum value (8.5 μg/day) of the adequate intake of vitamin D among all categories, so that all users can take vitamin D not less than the adequate intake.

In the example of FIGS. 1A and 1B, since the estimated energy requirement in the category of male, 18 to 29 years of age, and physical activity level III (high) is higher than in other categories, the amount of vitamin D that can be contained in the adjusted meal is 54.1 μg/day (100 μg×1650 kcal/3050 kcal), which is the lowest value. Accordingly, this value is specified as the upper limit of vitamin D intake in the adjusted meal.

Further, as another adjusting method, it is also preferable that vitamin D intake is determined within a range between the values obtained by defining the total number of calories taken in a day as 1650 kcal in the category with the lowest estimated energy requirement, i.e., the category of female, 50 to 64 years of age, and physical activity level I (low), in the Dietary Reference Intakes for Japanese for 18 to 64 years of age shown in FIGS. 1A and 1B and multiplying the above-mentioned upper limit and lower limit of vitamin D intake (8.5 μg/day and 54.1 μg/day) by the value obtained by dividing the number of calories taken in a meal with adjusted nutritional components to be eaten (breakfast, lunch, or evening meal) by the above-mentioned 1650 kcal.

Vitamin E

It is recommended to take vitamin E in consideration of balance, and vitamin E intake is preferably determined within a range between the values obtained by preferably setting the upper limit and the lower limit of daily vitamin D intake as described below and multiplying these upper limit and lower limit by the value obtained by dividing the number of calories taken in an adjusted meal to be eaten (breakfast, lunch, or evening meal) by the total number of calories designed to be taken in a day (i.e., percentage of calorie intake of each meal).

Here, the upper limit and the lower limit of vitamin E intake are calculated as descried below. In the “Dietary Reference Intakes for Japanese,” the adequate intake and the tolerable upper intake level are established as the Dietary Reference Intakes of vitamin E for 18 to 64 years of age.

Here, the amount of the adjusted meal of this embodiment is adjusted depending on the attributes of each user. Therefore, the amount of vitamin E contained in the adjusted meal with an adjusted amount needs to be less than the Dietary Reference Intakes for a category with high estimated energy requirement. Accordingly, a normalized vitamin E amount in each category is calculated by the following equation, and the upper limit of vitamin E intake in the adjusted meal is defined as the smallest normalized vitamin E amount among all.

Normalized vitamin E amount=tolerable upper intake level of vitamin E×(reference amount of energy/estimated energy requirement for each category)

Further, the lower limit of vitamin E intake in the adjusted meal is defined as the maximum value (7.0 mg/day) of the adequate intake of vitamin E among all categories, so that all users can take vitamin E not less than the adequate intake.

In the example of FIGS. 1A and 1B, since the estimated energy requirement in the category of male, 18 to 29 years of age, and physical activity level III (high) is higher than in other categories, the amount of vitamin E that can be contained in the adjusted meal is 459.8 mg/day (=850 mg×1650 kcal/3050 kcal), which is the lowest value. Accordingly, this value is specified as the upper limit of vitamin E intake in the adjusted meal.

Further, as another adjusting method, it is also preferable that vitamin E intake is determined within a range between the values obtained by defining the total number of calories taken in a day as 1650 kcal in the category with the lowest estimated energy requirement, i.e., the category of female, 50 to 64 years of age, and physical activity level I (low), in the Dietary Reference Intakes for Japanese for 18 to 64 years of age shown in FIGS. 1A and 1B and multiplying the above-mentioned upper limit and lower limit of vitamin E intake (7.0 mg/day and 459.8 mg/day) by the value obtained by dividing the number of calories taken in a meal with adjusted nutritional components to be eaten (breakfast, lunch, or evening meal) by the above-mentioned 1650 kcal.

Vitamin K

It is recommended to take vitamin K in consideration of balance, and vitamin K intake is preferably within a range not less than the value obtained by setting the lower limit of daily vitamin K intake as described below and multiplying the lower limit by the value obtained by dividing the number of calories taken in an adjusted meal to be eaten (breakfast, lunch, or evening meal) by the total number of calories designed to be taken in a day (i.e., percentage of calorie intake of each meal).

Here, the lower limit of vitamin K is calculated as described below. The lower limit of vitamin K intake in the adjusted meal is defined as the maximum value (150 μg/day) of the adequate intake of vitamin K among all categories, so that all users can take vitamin K not less than the adequate intake. Of note, since 150 μg/day is established as the adequate intake of vitamin K regardless of age, sex, or physical activity level in the “Dietary Reference Intakes for Japanese,” the lower limit of vitamin K intake in the adjusted meal is the adequate intake of vitamin K.

Further, as another adjusting method, it is also preferable that vitamin K intake is determined within a range not less than the value obtained by defining the total number of calories taken in a day as 1650 kcal in the category with the lowest estimated energy requirement, i.e., the category of female, 50 to 64 years of age, and physical activity level I (low), in the Dietary Reference Intakes for Japanese for 18 to 64 years of age shown in FIGS. 1A and 1B and multiplying the above-mentioned lower limit of vitamin K intake (150 μg/day) by the value obtained by dividing the number of calories taken in a meal with adjusted nutritional components to be eaten (breakfast, lunch, or evening meal) by the above-mentioned 1650 kcal.

Vitamin B1

It is recommended to take vitamin B1 in consideration of balance, and vitamin B1 intake is preferably within a range not less than a value obtained by setting the lower limit of daily vitamin B1 intake as described below and multiplying this lower limit by the value obtained by dividing the number of calories taken in an adjusted meal to be eaten (breakfast, lunch, or evening meal) by the total number of calories designed to be taken in a day (i.e., percentage of calorie intake of each meal).

Here, the lower limit of vitamin B1 is calculated as described below. In the “Dietary Reference Intakes for Japanese,” 1.1 to 1.4 mg/day is established as the recommended dietary allowance of vitamin B1 depending on age, sex, and physical activity level. In this embodiment, the lower limit of vitamin B1 intake in the adjusted meal of this embodiment is defined as the maximum value (1.4 mg/day) of the adequate intake of vitamin B1 among all categories, so that all users can take the adequate intake of vitamin B1. Consequently, even if the calorie intake in a meal of men aged 18 to 49 years old is limited to 1650 kcal/day, for example, for the purpose of dieting or the like, the Dietary Reference Intakes of 1.4 mg/day, which is the adequate intake of vitamin B1, can be achieved by taking the adjusted meal.

Further, as another adjusting method, it is also preferable that vitamin B1 intake is determined within a range not less than the value obtained by defining the total number of calories taken in a day as 1650 kcal in the category with the lowest estimated energy requirement, i.e., the category of female, 50 to 64 years of age, and physical activity level I (low), in the Dietary Reference Intakes for Japanese for 18 to 64 years of age shown in FIGS. 1A and 1B and multiplying the above-mentioned lower limit of vitamin B1 intake (1.4 mg/day) by the value obtained by dividing the number of calories taken in a meal with adjusted nutritional components to be eaten (breakfast, lunch, or evening meal) by the above-mentioned 1650 kcal.

Vitamin B2

It is recommended to take vitamin B2 in consideration of balance, and vitamin B2 intake is preferably within a range not less than the value obtained by setting the lower limit of daily vitamin B2 intake as described below and multiplying this lower limit by the value obtained by dividing the number of calories taken in an adjusted meal to be eaten (breakfast, lunch, or evening meal) by the total number of calories designed to be taken in a day (i.e., percentage of calorie intake of each meal).

Here, the lower limit of vitamin B2 is calculated as described below. In the “Dietary Reference Intakes for Japanese,” 1.2 to 1.6 mg/day is established as the recommended dietary allowance of vitamin B2 depending on age, sex, and physical activity level. In this embodiment, the lower limit of vitamin B2 intake in the adjusted meal of this embodiment is defined as the maximum value (1.6 mg/day) of the adequate intake of vitamin B2 among all categories, so that all users can take the adequate intake of vitamin B2. Consequently, even if the calorie intake in a meal of men aged 18 to 49 years old is limited to 1650 kcal/day, for example, for the purpose of dieting or the like, the Dietary Reference Intakes of 1.6 mg/day, which is the adequate intake of vitamin B2, can be achieved by taking the adjusted meal.

Further, as another adjusting method, it is also preferable that vitamin B2 intake is determined within a range not less than the value obtained by defining the total number of calories taken in a day as 1650 kcal in the category with the lowest estimated energy requirement, i.e., the category of female, 50 to 64 years of age, and physical activity level I (low), in the Dietary Reference Intakes for Japanese for 18 to 64 years of age shown in FIGS. 1A and 1B and multiplying the above-mentioned lower limit of vitamin B2 intake (1.6 mg/day) by the value obtained by dividing the number of calories taken in a meal with adjusted nutritional components to be eaten (breakfast, lunch, or evening meal) by the above-mentioned 1650 kcal.

Niacin

It is recommended to take niacin in consideration of balance, and niacin intake is preferably determined within a range between the values obtained by preferably setting the upper limit and the lower limit of daily niacin intake as described below and multiplying these upper limit and lower limit by the value obtained by dividing the number of calories taken in an adjusted meal to be eaten (breakfast, lunch, or evening meal) by the total number of calories designed to be taken in a day (i.e., percentage of calorie intake of each meal).

Here, the upper limit and the lower limit of niacin intake are calculated as described below. In the “Dietary Reference Intakes for Japanese,” the recommended dietary allowance and the tolerable upper intake level are established for niacin for 18 to 64 years of age. Here, the tolerable upper intake level is the tolerable upper intake level when niacin is taken as nicotinic acid derived from fortified food or supplement.

The amount of the adjusted meal of this embodiment is adjusted depending on the attributes of each user. Therefore, the amount of nicotinic acid contained in the adjusted meal with an adjusted amount needs to be less than the Dietary Reference Intakes for a category with high estimated energy requirement. Accordingly, a normalized nicotinic acid amount in each category is calculated by the following equation, and the upper limit of nicotinic acid intake in the adjusted meal is defined as the smallest normalized nicotinic acid amount among all.

Normalized nicotinic acid amount=tolerable upper intake level of nicotinic acid×(reference amount of energy/estimated energy requirement for each category)

Further, the lower limit of niacin intake as niacin equivalent in the adjusted meal is defined as the maximum value (15 mg NE/day) of the recommended dietary allowance of niacin equivalent among all categories, so that all users can take niacin equivalent not less than the recommended dietary allowance. In the example of FIGS. 1A and 1B, since the estimated energy requirement in the category of male, 18 to 29 years of age, and physical activity level III (high) is higher than in other categories, the amount of nicotinic acid that can be contained in the adjusted meal is 43.3 mg/day (80 mg×1650 kcal/3050 kcal), which is the lowest value.

Accordingly, this value is specified as the upper limit of nicotinic acid intake in the adjusted meal. Of note, in the “Dietary Reference Intakes for Japanese,” the upper limits of nicotinamide and nicotinic acid derived from fortified food or supplement are established, which is also applicable in this embodiment.

Further, as another adjusting method, it is also preferable that niacin intake is determined to be the value or more obtained by defining the total number of calories taken in a day as 1650 kcal in the category with the lowest estimated energy requirement, i.e., the category of female, 50 to 64 years of age, and physical activity level I (low), in the Dietary Reference Intakes for Japanese for 18 to 64 years of age shown in FIGS. 1A and 1B and multiplying the above-mentioned lower limit (15 mg NE) and the upper limit of niacin intake (43.3 mg/day) by the value obtained by dividing the number of calories taken in a meal with adjusted nutritional components to be eaten (breakfast, lunch, or evening meal) by the above-mentioned 1650 kcal.

Vitamin B6

It is recommended to take vitamin B6 in consideration of balance, and vitamin B6 intake is preferably determined within a range between the values obtained by preferably setting the upper limit and the lower limit of daily vitamin B6 intake as described below and multiplying these upper limit and lower limit by the value obtained by dividing the number of calories taken in an adjusted meal to be eaten (breakfast, lunch, or evening meal) by the total number of calories designed to be taken in a day (i.e., percentage of calorie intake of each meal).

Here, the upper limit and the lower limit of vitamin B6 intake are calculated as described below. In the “Dietary Reference Intakes for Japanese,” the recommended dietary allowance and the tolerable upper intake level are established as the Dietary Reference Intakes of vitamin B6 for 18 to 64 years of age.

Here, the amount of the adjusted meal of this embodiment is adjusted depending on the attributes of each user. Therefore, vitamin B6 contained in the adjusted meal with an adjusted amount needs to be less than the Dietary Reference Intakes for a category with high estimated energy requirement. Accordingly, a normalized vitamin B6 amount in each category is calculated by the following equation, and the upper limit of vitamin B6 intake in the adjusted meal is defined as the smallest normalized vitamin B6 amount among all.

Normalized vitamin B6 amount=tolerable upper intake level of vitamin B6×(reference amount of energy/estimated energy requirement for each category)

Further, the lower limit of vitamin B6 intake in the adjusted meal is defined as the maximum value (1.4 mg/day) of the recommended dietary allowance of vitamin B6 among all categories, so that all users can take vitamin B6 not less than the recommended dietary allowance.

In the example of FIGS. 1A and 1B, since the estimated energy requirement in the category of male, 18 to 29 years of age, and physical activity level III (high) is higher than in other categories, the amount of vitamin B6 that can be contained in the adjusted meal is 29.75 mg/day (55 mg×1650 kcal/3050 kcal), which is the lowest value. Accordingly, the upper limit of vitamin B6 intake in the adjusted meal is 29.75 mg. Consequently, even if users belonging to the category of male, 18 to 29 years of age, and physical activity level III (high) eat 3050 kcal/day by eating the adjusted meal with 1650 kcal/day containing vitamin B6 less than the above-mentioned upper limit of intake (e.g., 29.75 mg/day), the tolerable upper intake level of 55 mg/day for vitamin B6 would not be exceeded.

Further, as another adjusting method, it is also preferable that vitamin B6 intake is determined within a range between the values obtained by defining the total number of calories taken in a day as 1650 kcal in the category with the lowest estimated energy requirement, i.e., the category of female, 50 to 64 years of age, and physical activity level I (low), in the Dietary Reference Intakes for Japanese for 18 to 64 years of age shown in FIGS. 1A and 1B and multiplying the above-mentioned upper limit and lower limit of vitamin B6 intake (1.4 mg/day and 29.75 mg/day) by the value obtained by dividing the number of calories taken in a meal with adjusted nutritional components to be eaten (breakfast, lunch, or evening meal) by the above-mentioned 1650 kcal.

Vitamin B12

It is recommended to take vitamin B12 in consideration of balance, and vitamin B12 intake is preferably determined within a range not less than the value obtained by preferably setting the lower limit of daily vitamin B12 intake as described below and multiplying this lower limit by the value obtained by dividing the number of calories taken in an adjusted meal to be eaten (breakfast, lunch, or evening meal) by the total number of calories designed to be taken in a day (i.e., percentage of calorie intake of each meal).

Here, the lower limit of vitamin B12 intake is calculated as described above. In the “Dietary Reference Intakes for Japanese,” the recommended dietary allowance is established. In this embodiment, the lower limit of vitamin B12 intake in the adjusted meal is defined as the maximum value of vitamin B12 among all categories, so that all users can take the recommended dietary allowance of vitamin B12. Of note, since 2.4 μg/day is established as the recommended dietary allowance of vitamin B12 regardless of age, sex, or physical activity level in the “Dietary Reference Intakes for Japanese,” the lower limit of vitamin B12 intake in the adjusted meal is the recommended dietary allowance of vitamin B12.

Further, as another adjusting method, it is preferable that vitamin B12 intake is determined within a range not less than the value obtained by defining the total number of calories taken in a day as 1650 kcal in the category with the lowest estimated energy requirement, i.e., the category of female, 50 to 64 years of age, and physical activity level I (low), in the Dietary Reference Intakes for Japanese for 18 to 64 years of age shown in FIGS. 1A and 1B and multiplying the above-mentioned lower limit of vitamin B12 intake (2.4 μg/day) by the value obtained by dividing the number of calories taken in a meal with adjusted nutritional components to be eaten (breakfast, lunch, or evening meal) by the above-mentioned 1650 kcal.

Biotin

It is recommended to take biotin in consideration of balance, and biotin intake is preferably determined within a range not less than the value obtained by preferably setting the lower limit of daily biotin intake as described below and multiplying this lower limit by the value obtained by dividing the number of calories taken in an adjusted meal to be eaten (breakfast, lunch, or evening meal) by the total number of calories designed to be taken in a day (i.e., percentage of calorie intake of each meal).

Here, the lower limit of biotin intake is calculated as described below. In the “Dietary Reference Intakes for Japanese,” the adequate intake is established. In this embodiment, the lower limit of biotin intake in the adjusted meal is defined as the maximum value of the adequate intake of biotin among all categories, so that all users can take the adequate intake of biotin. Of note, since 50 μg/day is established as the adequate intake of biotin regardless of age, sex, or physical activity level in the “Dietary Reference Intakes for Japanese,” the lower limit of biotin intake in the adjusted meal is the adequate intake of biotin.

Further, as another adjusting method, it is preferable that biotin intake is determined within a range not less than the value obtained by defining the total number of calories taken in a day as 1650 kcal in the category with the lowest estimated energy requirement, i.e., the category of female, 50 to 64 years of age, and physical activity level I (low), in the Dietary Reference Intakes for Japanese for 18 to 64 years of age shown in FIGS. 1A and 1B and multiplying the above-mentioned lower limit of biotin intake (50 μg/day) by the value obtained by dividing the number of calories taken in a meal with adjusted nutritional components to be eaten (breakfast, lunch, or evening meal) by the above-mentioned 1650 kcal.

Vitamin C

It is recommended to take vitamin C in consideration of balance, and vitamin C intake is preferably determined within a range not less than the value obtained by preferably setting the lower limit of daily vitamin C intake as described below and multiplying this lower limit by the value obtained by dividing the number of calories taken in an adjusted meal to be eaten (breakfast, lunch, or evening meal) by the total number of calories designed to be taken in a day (i.e., percentage of calorie intake of each meal).

Here, the lower limit of vitamin C intake is calculated as described below. In the “Dietary Reference Intakes for Japanese,” the recommended dietary allowance is established. In this embodiment, the lower limit of vitamin C intake in the adjusted meal is defined as the maximum value of the recommended dietary allowance of vitamin C among all categories, so that all users can take the recommended dietary allowance of vitamin C. Of note, since 100 mg/day is established as the adequate intake of vitamin C regardless of age, sex, or physical activity level in the “Dietary Reference Intakes for Japanese,” the lower limit of vitamin C intake in the adjusted meal is the recommended dietary allowance of vitamin C.

Further, as another adjusting method, it is preferable that vitamin C intake is determined within a range not less than the value obtained by defining the total number of calories taken in a day as 1650 kcal in the category with the lowest estimated energy requirement, i.e., the category of female, 50 to 64 years of age, and physical activity level I (low), in the Dietary Reference Intakes for Japanese and multiplying the above-mentioned lower limit of vitamin C intake (100 mg/day) by the value obtained by dividing the number of calories taken in a meal with adjusted nutritional components to be eaten (breakfast, lunch, or evening meal) by the above-mentioned 1650 kcal.

Pantothenic Acid

It is recommended to take pantothenic acid in consideration of balance, and pantothenic acid intake is preferably determined within a range not less than the value obtained by preferably setting the lower limit of daily pantothenic acid intake as described below and multiplying this lower limit by the value obtained by dividing the number of calories taken in an adjusted meal to be eaten (breakfast, lunch, or evening meal) by the total number of calories designed to be taken in a day (i.e., percentage of calorie intake of each meal).

Here, the lower limit of pantothenic acid intake is calculated as described below. In the “Dietary Reference Intakes for Japanese,” 5 to 6 mg/day is established as the adequate intake of pantothenic acid depending on age, sex, and physical activity level. In this embodiment, the lower limit of pantothenic acid intake in the adjusted meal is defined as the maximum value (6 mg/day) of the adequate intake of pantothenic acid among all categories, so that all users can take the adequate intake of pantothenic acid. Consequently, all users can take the adequate intake of pantothenic acid when they eat at least the adjusted meal.

Further, as another adjusting method, it is also preferable that pantothenic acid intake is determined within a range not less than the value obtained by defining the total number of calories taken in a day as 1650 kcal in the category with the lowest estimated energy requirement, i.e., the category of female, 50 to 64 years of age, and physical activity level I (low), in the Dietary Reference Intakes for Japanese for 18 to 64 years of age shown in FIGS. 1A and 1B and multiplying the above-mentioned lower limit of pantothenic acid intake (6 mg/day) by the value obtained by dividing the number of calories taken in a meal with adjusted nutritional components to be eaten (breakfast, lunch, or evening meal) by the above-mentioned 1650 kcal.

Folic Acid

It is recommended to take folic acid in consideration of balance, and folic acid intake is preferably determined within a range between the values obtained by preferably setting the upper limit and the lower limit of daily folic acid intake as described below and multiplying these upper limit and lower limit by the value obtained by dividing the number of calories taken in an adjusted meal to be eaten (breakfast, lunch, or evening meal) by the total number of calories designed to be taken in a day (i.e., percentage of calorie intake of each meal).

Here, the upper limit and the lower limit of folic acid intake are calculated as described below. In the “Dietary Reference Intakes for Japanese,” the recommended dietary allowance and the tolerable upper intake level are established as the Dietary Reference Intakes of folic acid for 18 to 64 years of age.

Here, the amount of the adjusted meal of this embodiment is adjusted depending on the attributes of each user. Therefore, folic acid contained in the adjusted meal with an adjusted amount needs to be less than the Dietary Reference Intakes for a category with high estimated energy requirement. Accordingly, a normalized folic acid amount in each category is calculated by the following equation, and the upper limit of folic acid intake in the adjusted meal is defined as the smallest normalized folic acid amount among all.

Normalized folic acid amount=tolerable upper intake level of folic acid×(reference amount of energy/estimated energy requirement for each category)

Further, the lower limit of folic acid intake in the adjusted meal is defined as the maximum value (240 μg/day) of the recommended dietary allowance of folic acid among all categories, so that all users can take folic acid not less than the recommended dietary allowance.

In the example of FIGS. 1A and 1B, since the estimated energy requirement in the category of male, 18 to 29 years of age, and physical activity level III (high) is higher than in other categories, the amount of folic acid that can be contained in the adjusted meal is 486.9 μg/day (900 μg×1650 kcal/3050 kcal), which is the lowest value. Accordingly, the upper limit of folic acid intake in the adjusted meal is 486.9 μg/day.

Consequently, even if users belonging to the category of male, 18 to 29 years of age, and physical activity level III (high) eat 3050 kcal/day by eating the adjusted meal with 1650 kcal/day containing folic acid less than the above-mentioned upper limit of intake, the tolerable upper intake level of 900 μg/day for folic acid would not be exceeded.

Of note, in the “Dietary Reference Intakes for Japanese,” the upper limit is established only for pteroylmonoglutamic acid to be added, which is also applicable in this embodiment.

Further, as another adjusting method, it is also preferable that folic acid intake is determined within a range between the values obtained by defining the total number of calories taken in a day as 1650 kcal in the category with the lowest estimated energy requirement, i.e., the category of female, 50 to 64 years of age, and physical activity level I (low), in the Dietary Reference Intakes for Japanese for 18 to 64 years of age shown in FIGS. 1A and 1B and multiplying the above-mentioned upper limit and lower limit of folic acid intake (240 μg/day and 486.9 μg/day) by the value obtained by dividing the number of calories taken in a meal with adjusted nutritional components to be eaten (breakfast, lunch, or evening meal) by the above-mentioned 1650 kcal.

Further, it is recommended to review numerical values rationally for adjusted amounts of nutritional components in response to revisions of the Dietary Reference Intakes.

—Meal with Adjusted Nutritional Components—

For the above-described adjustment of nutritional components, a menu of vegetable, grain, meat, fish, and the like and processed food thereof, if necessary, cooked by a method such as broiling, simmering, steaming, or deep-frying, and, if necessary, seasoned with seasonings is provided as a fundamental breakfast, lunch, or evening meal. In addition, it is needless to say that various nutritional components that are inadequately taken can be supplemented by supplementarily utilizing nutritional supplementary foods, food additives, functional raw materials, various salts, and the like.

### —Continually Taking—

In the present invention, the above-described adjusted meal with adjusted vitamins and minerals needs to be continually taken. The term “continually taken” used in the present invention refers to taking the adjusted meal serially and over a predetermined period.

The term “continually” used herein can include various embodiments, and examples thereof include taking the adjusted meal every day. Also, examples include a method of taking the adjusted meal only on weekdays in one week (excluding the weekend). Further, a pattern of taking the adjusted meal on two days and not taking on one day is also possible. In addition, a method of taking the adjusted meal every other day is also considered.

Of note, a range of approximately 20% to 100% is acceptable as the frequency of taking the adjusted meal. A range of approximately 45% to 100% is further preferred. Further, a range of approximately 70% to 100% is more preferred.

As the duration of taking the adjusted meal, two to three weeks or longer is preferred for the method of taking the adjusted meal every day. Further, continually taking the adjusted meal for three weeks or longer is preferred for the method of taking the adjusted meal only on weekdays in one week (excluding Saturday and Sunday). Various taking methods are considered, and it is preferable to continually take the adjusted meal roughly for 15 days or longer, preferably for 17 to 19 days or longer.

Further, it is needless to say that it is acceptable to achieve continually taking the adjusted meal on the whole even if there are irregularly days when the adjusted meal is missed because of daily problems of eaters and the like.

In other words, it is sufficient to take the above-described adjusted meal for a predetermined duration.

### —Improving Health Conditions—

The term “improving health conditions” used in the present invention is defined as “health is a state of complete physical, mental and social well-being and not merely the absence of disease or infirmity” as defined in the Constitution of the World Health Organization.

Further, the conditions of a healthy individual generally include the following: absence of disease, good appetite and good bowel movement, being energetic and getting hardly tired, sufficient sleep, having resistance and being not susceptible to disease, good posture and physical harmony, and normal development.

Such health conditions are assessed with various health indicators. Specific examples of health indicators include body weight, BMI, body fat percentage, blood triglyceride, LDL-cholesterol, blood glucose, blood pressure, bone density, presenteeism, QOL, mood state, fatigue, stress, bowel movement, intestinal flora, sleep status, life expectancy, and prevalence.

In particular, in the present invention, the health conditions that are assessed with one or more health indicators selected from blood triglyceride, bone density, presenteeism, oxidative stress marker, and intestinal flora diversity can be improved by continually taking a meal with adjusted nutritional components.

### Examples

Hereinafter an example of the present invention will be described. The present invention is not limited to the following example.

### <Study Method>

To investigate change in the awareness of physical conditions and health after taking a “healthy meal” in which a plurality of nutritional components considered good for health were adjusted and mixed for breakfast and lunch, as compared with before taking the meal, an open study was conducted in healthy adult men and women (aged 37.4±8.8 years old). Details are described below.

### —Subjects—

A total of 83 subjects (67 men and 16 women) participated in this study.

—Duration when the Meal with Adjusted Nutritional Components was Taken—

The duration when the adjusted meal was taken was 17 to 19 days (approximately four weeks). The meal with adjusted nutritional components was taken only on weekdays in one week, and meals were eaten freely on weekends and public holidays.

—Time of Taking the Meal with Adjusted Nutritional Components in Terms of Daily Three Meals (Breakfast, Lunch, and Evening Meal)—

During the period when the meal with adjusted nutritional components was taken, the meal was taken only for breakfast and lunch out of daily three meals (breakfast, lunch, and evening meal), and other meals (evening meal and snack) were eaten freely.

Five types of meals with adjusted nutritional components were prepared for breakfast, and 15 types of meals were prepared for lunch. Subjects chose and took one type from the five types of meals for breakfast on days when the staff canteen was open (i.e., on business days) and chose either of two types provided from the 15 types of meals for lunch at the staff canteen.

### —Tests and Subjects for Analysis—

Only subjects who took 80% or more of the meals with adjusted nutritional components during the above-mentioned meal ingestion period were to be included in the analysis. As a result, 75 subjects (62 men, 13 women) were included in the analysis. Further, the data were analyzed using a “paired t test” and a “Wilcoxon signed rank sum test” with a significance level of 0.05.

—Meal with Adjusted Nutritional Components—

The following adjustments were made for the meal with adjusted nutritional components.
The total number of calories in each of breakfast and lunch was adjusted for each menu as follows because it varies depending on the menu of the adjusted meal.

Energy

Energy was adjusted in a range of roughly 280 to 340 kcal for breakfast and in a range of roughly 460 to 540 kcal for lunch.

PFC Balance

For the PFC balance, meals were adjusted so that each meal with adjusted nutritional components contains 13% to 20% of proteins, 20% to 30% of fats, and 50% to 65% of carbohydrates in terms of the energy percentage.

Proteins

As described above, each meal was adjusted so that protein intake should be within a range between the values obtained by defining the number of calories taken in a day as 1650 kcal in the category with the lowest estimated energy requirement, i.e., the category of female, 50 to 64 years of age, and physical activity level I (low), in the Dietary Reference Intakes for Japanese for 18 to 64 years of age shown in FIGS. 1A and 1B and multiplying the above-described upper limit and lower limit of protein intake (82.5 g/day and 60 g/day) by the value obtained by dividing the number of calories taken in each adjusted meal for breakfast and lunch by this 1650 kcal.

Fats

As described above, each meal was adjusted so that fat intake should be within a range between the values obtained by defining the number of calories taken in a day as 1650 kcal in the category with the lowest estimated energy requirement, i.e., the category of female, 50 to 64 years of age, and physical activity level I (low), in the Dietary Reference Intakes and multiplying the above-described upper limit and lower limit of fat intake (55.0 g/day and 36.7 g/day) by the value obtained by dividing the number of calories taken in each adjusted meal for breakfast and lunch by this 1650 kcal.

It is preferable to set the upper limit of saturated fatty acid intake. As in the case of fats, each meal was adjusted so that saturated fatty acid intake should be within a range not more than the value obtained by multiplying the above-described upper limit of saturated fatty acid intake (12.8 g/day) by the value obtained by dividing the number of calories taken in each adjusted meal for breakfast and lunch by the above-mentioned 1650 kcal.

Further, it is preferable to set the lower limit of n-6 fatty acid intake. As in the case of fats, each meal was adjusted so that n-6 fatty acid intake should be within a range not less than the value obtained by multiplying the above-described lower limit of n-6 fatty acid intake (11 g/day) by the value obtained by dividing the number of calories taken in each adjusted meal for breakfast and lunch by the above-mentioned 1650 kcal.

Further, it is preferable to set the lower limit of n-3 fatty acid intake. As in the case of fats, each meal was adjusted so that n-3 fatty acid intake should be within a range not less than the value obtained by multiplying the above-described lower limit of n-3 fatty acid intake (2.2 g/day) by the value obtained by dividing the number of calories taken in each adjusted meal for breakfast and lunch by the above-mentioned 1650 kcal.

Carbohydrates

As described above, each meal was adjusted so that carbohydrate intake should be within a range between the values obtained by defining the number of calories taken in a day as 1650 kcal in the category with the lowest estimated energy requirement, i.e., the category of female, 50 to 64 years of age, and physical activity level I (low), in the Dietary Reference Intakes for Japanese and multiplying the above-described upper limit and lower limit of carbohydrate intake (268.1 g/day and 206.3 g/day) by the value obtained by dividing the number of calories taken in each adjusted meal for breakfast and lunch by the above-mentioned 1650 kcal.

Further, as described above, each meal was also adjusted so that dietary fiber intake should be within a range not less than the value obtained by multiplying the above-described lower limit of dietary fiber intake (21 g/day) by the value obtained by dividing the number of calories taken in each adjusted meal for breakfast and lunch by the above-mentioned 1650 kcal.

Minerals and Vitamins

Sodium (Salt Equivalent)

Each meal was adjusted so that sodium intake (salt equivalent) should be 1.7 g or less for breakfast and less than 3.0 g for lunch.

Calcium

As described above, each meal was adjusted so that calcium intake should be within a range between the values obtained by multiplying the above-described upper limit and lower limit of calcium intake (1352.5 mg/day and 800 mg/day) by the value obtained by dividing the number of calories taken in each adjusted meal for breakfast and lunch by the above-mentioned 1650 kcal.

Iron

As described above, each meal was adjusted so that iron intake should be within a range between the values obtained by multiplying the above-described upper limit and lower limit of iron intake (27.0 mg/day and 10.5 mg/day) by the value obtained by dividing the number of calories taken in each adjusted meal for breakfast and lunch by the above-mentioned 1650 kcal.

Phosphorus

As described above, each meal was adjusted so that phosphorus intake should be within a range between the values obtained by multiplying the above-described upper limit and lower limit of phosphorus intake (1623 mg/day and 1000 mg/day) by the value obtained by dividing the number of calories taken in each adjusted meal for breakfast and lunch by the above-mentioned 1650 kcal.

Magnesium

As described above, each meal was adjusted so that magnesium intake should be within a range not less than the value obtained by multiplying the above-described lower limit of magnesium intake (370 mg/day) by the value obtained by dividing the number of calories taken in each adjusted meal for breakfast and lunch by the above-mentioned 1650 kcal.

Potassium

As described above, each meal was adjusted so that potassium intake should be within a range not less than the value obtained by multiplying the above-described lower limit of potassium intake (2500 mg/day) by the value obtained by dividing the number of calories taken in each adjusted meal for breakfast and lunch by the above-mentioned 1650 kcal.

Copper

As described above, each meal was adjusted so that copper intake should be within a range between the values obtained by multiplying the above-described upper limit and lower limit of copper intake (3.79 mg/day and 0.9 mg/day) by the value obtained by dividing the number of calories taken in each adjusted meal for breakfast and lunch by the above-mentioned 1650 kcal.

Iodine

As described above, each meal was adjusted so that iodine intake should be within a range between the values obtained by multiplying the above-described upper limit and lower limit of iodine intake (1623 μg/day and 130 μg/day) by the value obtained by dividing the number of calories taken in each adjusted meal for breakfast and lunch by the 1650 kcal.

Selenium

As described above, each meal was adjusted so that selenium intake should be within a range between the values obtained by multiplying the above-described upper limit and lower limit of selenium intake (243.4 μg/day and 30 μg/day) by the value obtained by dividing the number of calories taken in each adjusted meal for breakfast and lunch by the above-mentioned 1650 kcal.

Zinc

As described above, each meal was adjusted so that zinc intake should be within a range between the values obtained by multiplying the above-described upper limit and lower limit of selenium intake (21.6 mg/day and 11 mg/day) by the value obtained by dividing the number of calories taken in each adjusted meal for breakfast and lunch by the above-mentioned 1650 kcal.

Chromium

As described above, each meal was adjusted so that chromium intake should be within a range between the values obtained by multiplying the above-described upper limit and lower limit of chromium intake (270.5 μg/day and 10 μg/day) by the value obtained by dividing the number of calories taken in each adjusted meal for breakfast and lunch by the above-mentioned 1650 kcal.

Manganese

As described above, each meal was adjusted so that manganese intake should be within a range between the values obtained by multiplying the above-described upper limit and lower limit of manganese intake (6.0 mg/day and 4.0 mg/day) by the value obtained by dividing the number of calories taken in each adjusted meal for breakfast and lunch by the above-mentioned 1650 kcal.

Molybdenum

As described above, each meal was adjusted so that molybdenum intake should be within a range between the values obtained by multiplying the above-described upper limit and lower limit of molybdenum intake (324.6 μg/day and 30 μg/day) by the value obtained by dividing the number of calories taken in each adjusted meal for breakfast and lunch by the above-mentioned 1650 kcal.

Vitamin A

As described above, each meal was adjusted so that vitamin A intake should be within a range between the values obtained by multiplying the above-described upper limit and lower limit of vitamin A intake (1460.7 μg RE/day and 900 μg RE/day) by the value obtained by dividing the number of calories taken in each adjusted meal for breakfast and lunch by the above-mentioned 1650 kcal.

Vitamin D

As described above, each meal was adjusted so that vitamin D intake should be within a range between the values obtained by multiplying the above-described upper limit and lower limit of vitamin D intake (54.1 μg/day and 8.5 μg/day) by the value obtained by dividing the number of calories taken in each adjusted meal for breakfast and lunch by the above-mentioned 1650 kcal.

Vitamin E

As described above, each meal was adjusted so that vitamin E intake should be within a range between the values obtained by multiplying the above-described upper limit and lower limit of vitamin E intake (459.8 mg/day and 7.0 mg/day) by the value obtained by dividing the number of calories taken in each adjusted meal for breakfast and lunch by the above-mentioned 1650 kcal.

Vitamin K

As described above, each meal was adjusted so that vitamin K intake should be within a range not less than the value obtained by multiplying the above-described lower limit of vitamin K intake (150 μg/day) by the value obtained by dividing the number of calories taken in each adjusted meal for breakfast and lunch by the above-mentioned 1650 kcal.

Vitamin B1

As described above, each meal was adjusted so that vitamin B1 intake should be within a range not less than the value obtained by multiplying the above-described lower limit of vitamin B1 intake (1.4 mg/day) by the value obtained by dividing the number of calories taken in each adjusted meal for breakfast and lunch by the above-mentioned 1650 kcal.

Vitamin B2

As described above, each meal was adjusted so that vitamin B2 intake should be within a range not less than the value obtained by multiplying the above-described lower limit of vitamin B2 intake (1.6 mg/day) by the value obtained by dividing the number of calories taken in each adjusted meal for breakfast and lunch by the above-mentioned 1650 kcal.

Niacin

As described above, each meal was adjusted so that niacin intake should be within a range between the values obtained by multiplying the above-described upper limit and lower limit of niacin intake (43.3 mg/day and 15 mg NE/day) by the value obtained by dividing the number of calories taken in each adjusted meal for breakfast and lunch by the above-mentioned 1650 kcal.

Vitamin B6

As described above, each meal was adjusted so that vitamin B6 should be within a range between the values obtained by multiplying the above-described upper limit and lower limit of vitamin B6 intake (29.75 mg/day and 1.4 mg/day) by the value obtained by dividing the number of calories taken in each adjusted meal for breakfast and lunch by the above-mentioned 1650 kcal.

Vitamin B12

As described above, each meal was adjusted so that vitamin B12 intake should be within a range not less than the value obtained by multiplying the above-described lower limit of vitamin B12 intake (2.4 μg/day) by the value obtained by dividing the number of calories taken in each adjusted meal for breakfast and lunch by the above-mentioned 1650 kcal.

Biotin

As described above, each meal was adjusted so that biotin intake should be within a range not less than the value obtained by multiplying the above-described lower limit of biotin intake (50 μg/day) by the value obtained by dividing the number of calories taken in each adjusted meal for breakfast and lunch by the above-mentioned 1650 kcal.

Vitamin C

As described above, each meal was adjusted so that vitamin C intake should be within a range not less than the value obtained by multiplying the above-described lower limit of vitamin C intake (100 mg/day) by the value obtained by dividing the number of calories taken in each adjusted meal for breakfast and lunch by the above-mentioned 1650 kcal.

Pantothenic Acid

As described above, each meal was adjusted so that pantothenic acid intake should be within a range not less than the value obtained by multiplying the above-described lower limit of pantothenic acid intake (6 mg/day) by the value obtained by dividing the number of calories taken in each adjusted meal for breakfast and lunch by the above-mentioned 1650 kcal.

Folic Acid

As described above, each meal was adjusted so that folic acid is within a range between the values obtained by multiplying the above-described upper limit and lower limit of folic acid intake (486.9 μg/day and 240 μg/day) by the value obtained by dividing the number of calories taken in each adjusted meal for breakfast and lunch by the above-mentioned 1650 kcal.

Other Considerations

For the above-described meal with adjusted nutritional components, a menu of vegetable, grain, meat, fish, and the like and processed food thereof, if necessary, cooked by a method such as broiling, simmering, steaming, or deep-frying, and, if necessary, seasoned with seasonings was provided as a fundamental menu. In addition, various nutritional components that are inadequately taken were supplemented by supplementarily utilizing nutritional supplementary foods, food additives, functional raw materials, various salts, and the like.

### <Results>

### (1) Physical Measurements

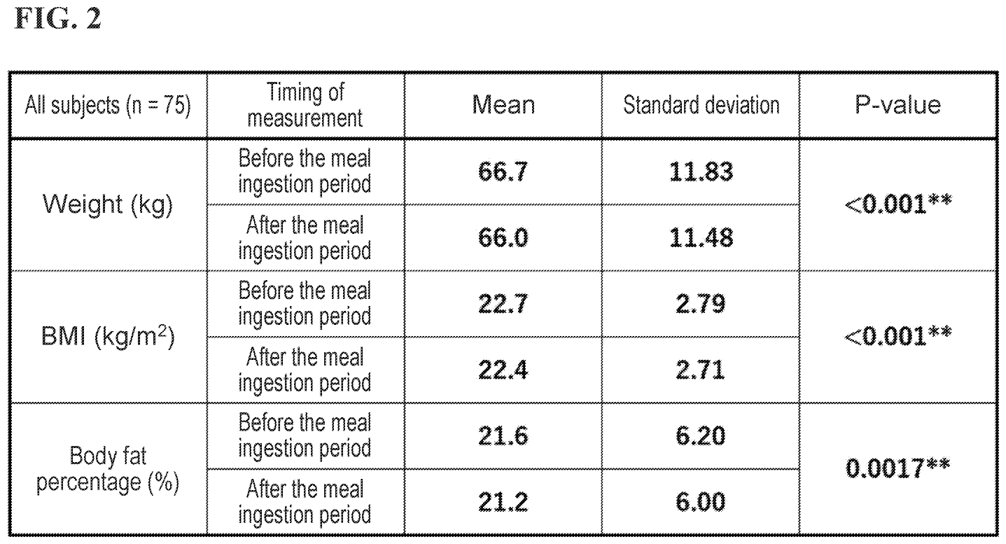

FIG. 2 shows the results of physical measurements before and after taking the above-described adjusted meal for a predetermined period. The body weight, BMI, and body fat percentage significantly decreased as compared with those before the meal ingestion period.

### (2) Blood Triglyceride

FIG. 3 below shows the results of analysis of triglyceride among blood lipids before and after taking the above-described adjusted meal for a predetermined period.

Blood triglyceride levels significantly decreased as compared with those before the meal ingestion period. The magnitude of the decrease appears to be greater in groups with higher triglyceride levels. Of note, hyperglyceridemia is determined if the baseline value in the above FIG. 3 is 150 or higher, and a high normal range is determined if it is 120 to 149.

### (3) Blood Metabolome Analysis

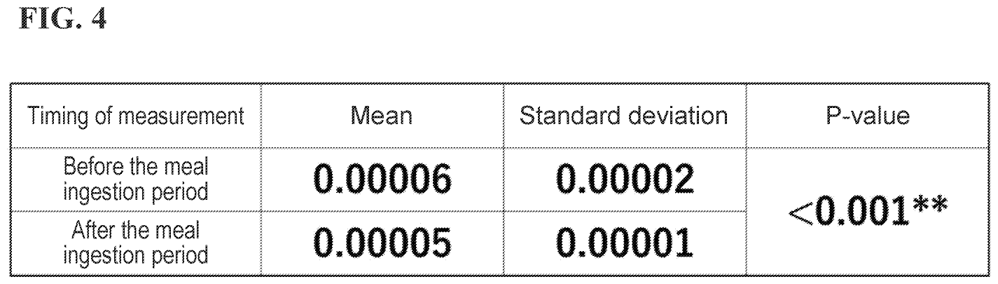

To analyze blood metabolome before and after taking the above-described adjusted meal for a predetermined period, blood (plasma) metabolites in subjects were analyzed using a capillary-electrophoresis-time-of-flight mass spectrometer (CE-TOFMS), and the peak areas of the detected 229 candidate compounds before and after taking the above-described adjusted meal for a predetermined period were compared. Further, of the 40 compounds that showed a significant change, those with evidence of effects on humans were investigated. As shown in FIG. 4, 8-hydroxy-2′-deoxyguanosine (8-OHdG) significantly decreased after the meal ingestion period. Of note, 8-OHdG is a compound with evidence of serving as an oxidative stress marker of DNA damage. Of note, FIG. 4 shows relative areas.

### (4) Blood Pressure

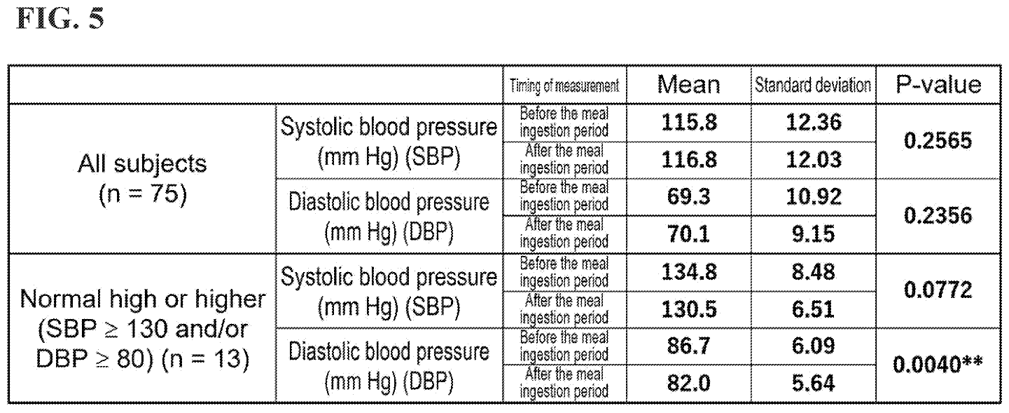

Blood pressure was measured before and after taking the above-described adjusted meal for a predetermined period. FIG. 5 shows the results. Further, a stratified analysis was also performed for a group with high normal or higher blood pressure.

In the group with high normal or higher blood pressure, systolic blood pressure tended to decrease, and diastolic blood pressure significantly decreased after the meal ingestion period.

### (5) Bone Density

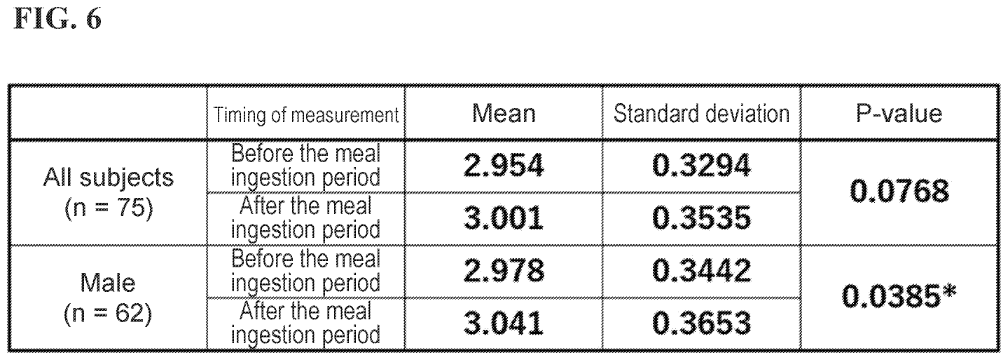

Bone density was measured before and after taking the above-described adjusted meal for a predetermined period. FIG. 6 shows the results. Of note, bone density was measured by an osteo-sono assessment index (OSI) using AOS-100SA (Hitachi, Ltd.).

After the meal ingestion period, bone density tended to increase overall and significantly increased in men.

### (6) Intestinal Flora Analysis

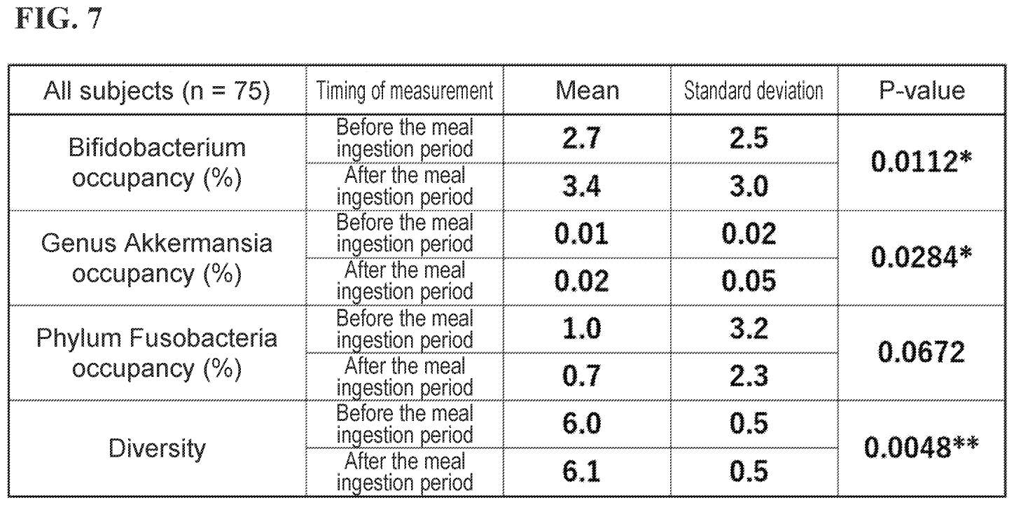

Intestinal flora was analyzed before and after taking the above-described adjusted meal for a predetermined period. FIG. 7 shows the results.

Improvement was observed in the intestinal flora indicators that are considered to have effects on health. The occupancy and diversity score of genera Bifidobacterium and Akkermansia significantly increased. Further, the occupancy of phylum Fusobacteria tended to decrease.

This application is based on Japanese Patent Application No. 2020-166818 applied on Oct. 1, 2020, of which content is incorporated herein by reference.

1. A method for improving health conditions by continually taking at least one meal selected from daily three meals, i.e., breakfast, lunch, and evening meal, which contains 13% to 20% of proteins, 20% to 30% of fats, and 50% to 65% of carbohydrates in terms of energy percentage and in which proteins, fats, carbohydrates, dietary fiber, vitamins, and minerals are adjusted to predetermined amounts.

2. The method for improving health conditions according to claim 1, wherein the health conditions are assessed by any one or more health indicators selected from body weight, BMI, body fat percentage, blood pressure, blood triglyceride, bone density, presenteeism, oxidative stress marker, stool frequency, and intestinal flora diversity.

3. The method for improving health conditions according to claim 1, wherein the vitamins include at least vitamin B1 and vitamin C, and the minerals include at least Ca, Mg, and Fe.

4. The method for improving health conditions according to claim 1, wherein the vitamins include at least vitamin A, vitamin B1, vitamin B2, vitamin B6, folic acid, and vitamin C, and the minerals include at least K, Ca, Mg, P, and Fe.

5. The method for improving health conditions according to claim 1, wherein the fats include saturated fatty acids not more than a predetermined amount and n-3 fatty acids and n-6 fatty acids not less than predetermined amounts.

6. The method for improving health conditions according to claim 1, wherein the meal is taken continually only on weekdays.

7. The method for improving health conditions according to claim 1, wherein the meal is taken continually for at least 3 weeks.

8. A meal with adjusted nutritional components to be taken in the method for improving health conditions according to claim 1.

9. The meal according to claim 8, wherein the meal is at least one meal selected from the group consisting of breakfast, lunch, and evening meal.

10. The method for improving health conditions according to claim 2, wherein the vitamins include at least vitamin B1 and vitamin C, and the minerals include at least Ca, Mg, and Fe.

11. The method for improving health conditions according to claim 2, wherein the vitamins include at least vitamin A, vitamin B1, vitamin B2, vitamin B6, folic acid, and vitamin C, and the minerals include at least K, Ca, Mg, P, and Fe.

12. The method for improving health conditions according to claim 2, wherein the fats include saturated fatty acids not more than a predetermined amount and n-3 fatty acids and n-6 fatty acids not less than predetermined amounts.

13. The method for improving health conditions according to claim 3, wherein the fats include saturated fatty acids not more than a predetermined amount and n-3 fatty acids and n-6 fatty acids not less than predetermined amounts.

14. The method for improving health conditions according to claim 4, wherein the fats include saturated fatty acids not more than a predetermined amount and n-3 fatty acids and n-6 fatty acids not less than predetermined amounts.

15. The method for improving health conditions according to claim 10, wherein the fats include saturated fatty acids not more than a predetermined amount and n-3 fatty acids and n-6 fatty acids not less than predetermined amounts.

16. The method for improving health conditions according to claim 11, wherein the fats include saturated fatty acids not more than a predetermined amount and n-3 fatty acids and n-6 fatty acids not less than predetermined amounts.

17. The method for improving health conditions according to claim 2, wherein the meal is taken continually only on weekdays.

18. The method for improving health conditions according to claim 3, wherein the meal is taken continually only on weekdays.

19. The method for improving health conditions according to claim 4, wherein the meal is taken continually only on weekdays.

20. The method for improving health conditions according to claim 5, wherein the meal is taken continually only on weekdays.
